(19) United States
(12) Patent Application Publication
(10) Pub. No.: US 2016/0297829 A1
(43) Pub. Date: Oct. 13, 2016

(54) METHOD FOR PRODUCING AVENACIOLIDES AND USES THEREOF

(71) Applicant: Academia Sinica, Taipei (TW)

(72) Inventors: Shih-Hsiung WU, Taipei (TW); Ching-Ming CHANG, Taipei (TW)

(21) Appl. No.: 14/681,553

(22) Filed: Apr. 8, 2015

(51) Int. Cl. C07D 493/08; C07D 307/33; A61K 45/06; A61K 31/365

(52) U.S. Cl. CPC C07D 493/08; A61K 31/365; C07D 307/33; A61K 45/06

(57) ABSTRACT

Disclosed herein are novel uses of avenaciolide derivatives and the preparation method of producing the same. The avenaciolide derivatives may suppress or inhibit the growth of gram-positive bacteria, including the notorious methicillin-resistant Staphylococcus aureus. Accordingly, the avenaciolides derivatives are potential lead compounds for the development of next generation antibiotics for the treatment of disease and/or disorders related to infection caused by gram-positive bacteria.

### BACKGROUND OF THE INVENTION

1. Field of the Invention

The present disclosure relates in general, to the field of antibacterials, and to the treatment of disorders caused by bacterial infection. More specifically, the present disclosure relates to novel uses of derivatives of avenaciolides, and methods for producing the same.

2. Description of Related Art

Antibacterial resistance is a global clinical and public health problem that is rising in an alarmingly speed. Physicians are now confronted with infections for which there is no effective therapy. The morbidity, mortality and financial costs of such infections pose an increasing burden on health care system worldwide, especially in countries with limited resources.

Gram-positive bacteria are those that are stained dark blue or violet by Gram staining. Gram-positive bacteria are generally characterized in having, as is part of their cell wall structure, peptidoglycan and polysaccharides. In general, six gram-positive bacteria are regarded as pathogenic in humans. Among them, Streptococcus, Staphylococcus and Enterococcus are the most notorious microorganisms, with various strains become drug-resistant. Examples of these drug-resistant strains include methicillin-resistant and/or vancomycin resistant strains, such as methicillin-resistant Staphylococcus aureus (MRSA), and vancomycin resistant S. aureus. The heavy use of vancomycin to treat MRSA infection has in turn contributed to the emergence of new strains of Enterococci. Enterococcus is known to be the cause of meningitis, and infections in the urinary tract, stomach and intestines. Infection caused by these vancomycin resistant Enterococci frequently do not respond to any current therapies, and in many cases prove fetal.

In view of the foregoing, there exists in this art a need of a novel anti-bacteria agent that may suppress the growth of gram-positive bacteria, particularly, the growth of drug-resistant gram-positive bacteria.

### SUMMARY

The present disclosure is based, at least in part, unexpected discovery that some derivatives of avenaciolide are effective in suppressing the growth of gram-positive bacteria, particularly, the drug-resistant gram-positive bacteria.

Accordingly, these avenaciolide derivatives are potential candidates as the lead compounds for the development of a medicament suitable for treating a disease associated with an infection caused by gram-positive bacteria, such as pneumonia, sepsis, cornea infection, skin infection, an infection in the central neuron system, or a toxic shock syndrome.

Accordingly, it is the first aspect of the present disclosure to provide a method of treating disease associated with a gram-positive bacterial infection in a subject, comprising administering to the subject, an effective amount of a derivative of avenaciolide, so as to alleviate or ameliorate the symptoms of the disease, wherein the derivative of avenaciolide has the structure of formula (I) or (II),

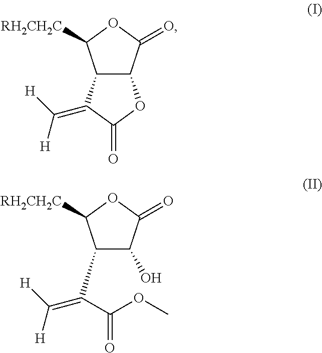

wherein, R is C₂₋₁₀ alkyl or C₂₋₁₀ alkenyl.

According to certain embodiments, R of the compound of formula (I) or (II) is C₃₋₆ alkyl. In a preferred example, R is n-hexyl.

According to some embodiments of the present disclosure, the gram-positive bacteria is any of Bacillus anthracis, Bacillus subtilis, Bacillus cereus, Corynebacterium diptheriae, Clostridium tetani, Clostridium botulinum, Clostridium perfringes, Clostridium difficile, Clostridium scindens, Enterococcim Streptococcus viridians, Enterococcus faecalis, Erysipelothrix rhusiopathiae, Escherichia Coli, Listeria monocytogens, Propionbacterium acnes, Rhodococcus equi, Staphylococcus agalactiae, Staphylococcus aureus, Staphylococcus epidermidis, Staphylococcus pneumonia, Staphylococcus pyrogens, or Staphylococcus saprophyticus.

According to certain embodiment, the gram-positive bacteria is Staphylococcus aureus. In one example, the Staphylococcus aureus is a methicillin-resistant Staphylococcus aureus (MRSA). According to a further example, the Staphylococcus aureus is a vancomycin resistant Staphylococcus aureus.

According to another embodiment, the gram-positive bacteria is Enterococcus faecalis. In one example, the Enterococcus faecalis is a vancomycin resistant Enterococcus faecalis.

According to some embodiments of the present disclosure, the disease is pneumonia, sepsis, cornea infection, skin infection, an infection in the central neuron system, or a toxic shock syndrome.

According to other embodiments, the subject has skin abscess, furuncle or skin boil.

According to some embodiments of the present disclosure, the method further comprises administering to the subject another therapeutic agent (e.g., an antibiotic) concurrently with, before and/or after administering the derivative of avenaciolide having the structure of formula (I) or (II), so as to alleviate or ameliorate the symptoms of the disease.

According to some preferred embodiments, examples of the antibiotic that may be used with the present method include, but are not limited to, acumycin, ampicillin, amoxycillin, amphotericins, antimycins, anglomycin, avermectins, azithromycin, boromycin, carbomycins, carbapenem, ceftazidime, cethromycin, chloramphenicol, chalcomycin, ciprofloxacin, concanamycins, cirramycin, clarithromycin, colistin, cycloxacillin, daptomycin, desmethyl azithromycin, desertomycins, dihydropikromycin, dirithromycin, doxycycline, enramycin, erythromycin, flurithromycin, flumequin gentamycin, juvenimicins, kujimycins, lankamycins, lincomycin, litorin, leucomycins, megalomicins, meropenem, methymycin, midecamycins, mycinamicin I, mycinamicin II, mycinamicin III, mycinamicin IV, mycinamicin V, mycinamicin VI, mycinamicin VII, mycinamicin VIII, narbomycin, neoantimycin, neomethymycin, netilmicin, neutromycin, niddamycins, norfioxacin, oleandomycins, oligomycins, ossamycin, oxacillin, oxolinic acid, penicillin, pikromycin, piperacillin, platenomycins, rapamycins, relomycin, rifamycins, rosaramicin, roxithromycin, virginiamycin, spiramycin, sporeamycin, staphococcomycin, streptomycin, sulfamethoxazole, swalpamycin, telithromycin, teicoplanin, timentin, tobramycin, ticarcillin, trimethoprim, tetracyclin, zlocillin, and/or a combination thereof.

Accordingly, it is the second aspect of the present disclosure to provide a composition that suppresses the growth of a gram-positive bacteria. The composition is therefore useful for treating a disease associated with a gram-positive bacterial infection. The composition comprises an effective amount of the afore-described compound of formula (I) or (II); and a pharmaceutically acceptable excipient.

The compound of formula (I) or (II) is present in the composition about 0.1% to 99% by weight, based on the total amount of the composition. In certain embodiments, the compound of formula (I) or (II) is present in the composition at least about 1% by weight, based on the total amount of the composition. In other embodiments, the compound of formula (I) or (II) is present in the composition at least about 5% by weight, based on the total amount of the composition. In further embodiments, the compound of formula (I) or (II) is present in the composition at least about 10% by weight, based on the total amount of the composition. In still further embodiments, the compound of formula (I) or (II) is present in the composition at least about 25% by weight, based on the total amount of the composition.

According to some preferred embodiments, the composition further comprises an antibiotic. Examples of the antibiotic that may be used with the present composition include, but are not limited to, acumycin, ampicillin, amoxycillin, amphotericins, antimycins, anglomycin, avermectins, azithromycin, boromycin, carbomycins, carbapenem, ceftazidime, cethromycin, chloramphenicol, chalcomycin, ciprofloxacin, concanamycins, cirramycin, clarithromycin, colistin, cycloxacillin, daptomycin, desmethyl azithromycin, desertomycins, dihydropikromycin, dirithromycin, doxycycline, enramycin, erythromycin, flurithromycin, flumequin gentamycin, juvenimicins, kujimycins, lankamycins, lincomycin, litorin, leucomycins, megalomicins, meropenem, methymycin, midecamycins, mycinamicin I, mycinamicin II, mycinamicin III, mycinamicin IV, mycinamicin V, mycinamicin VI, mycinamicin VII, mycinamicin VIII, narbomycin, neoantimycin, neomethymycin, netilmicin, neutromycin, niddamycins, norfioxacin, oleandomycins, oligomycins, ossamycin, oxacillin, oxolinic acid, penicillin, pikromycin, piperacillin, platenomycins, rapamycins, relomycin, rifamycins, rosaramicin, roxithromycin, virginiamycin, spiramycin, sporeamycin, staphococcomycin, streptomycin, sulfamethoxazole, swalpamycin, telithromycin, teicoplanin, timentin, tobramycin, ticarcillin, trimethoprim, tetracyclin, zlocillin, and/or a combination thereof.

Accordingly, it is the third aspect of the present disclosure to provide a method of suppressing the growth of a gram-positive bacteria comprising contacting the gram-positive bacteria with a sufficient amount of a derivative of avenaciolide having the structure of formula (I) or (II),

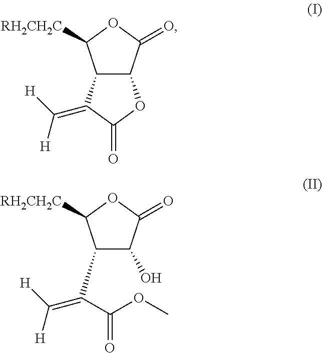

wherein, R is C₂₋₁₀ alkyl or C₂₋₁₀ alkenyl.

According to certain embodiments, R of the compound of formula (I) or (II) is C₃₋₆ alkyl. In a preferred example, R is n-hexyl.

According to some embodiments of the present disclosure, the gram-positive bacteria is any of Bacillus anthracis, Bacillus subtilis, Bacillus cereus, Corynebacterium diptheriae, Clostridium tetani, Clostridium botulinum, Clostridium perfringes, Clostridium difficile, Clostridium scindens, Enterococcim Streptococcus viridians, Enterococcus faecalis, Erysipelothrix rhusiopathiae, Escherichia Coli, Listeria monocytogens, Propionbacterium acnes, Rhodococcus equi, Staphylococcus agalactiae, Staphylococcus aureus, Staphylococcus epidermidis, Staphylococcus pneumonia, Staphylococcus pyrogens, or Staphylococcus saprophyticus.

According to certain embodiment, the gram-positive bacteria is Staphylococcus aureus. In one example, the Staphylococcus aureus is a methicillin-resistant Staphylococcus aureus (MRSA). According to a further example, the Staphylococcus aureus is a vancomycin resistant Staphylococcus aureus.

It is the fourth aspect of the present disclosure to provide a method of producing a compound of formula (I) or (II),

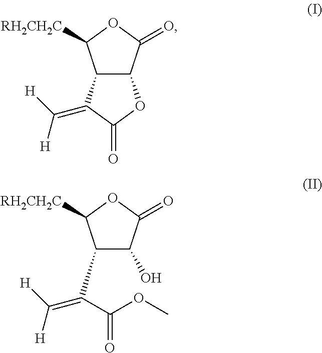

wherein R is C₂₋₁₀ alkyl or C₂₋₁₀ alkenyl, and the method comprises:
(1) using diacetone D-glucose as a starting material to produce compound 5;
(2) allowing the compound 5 to react with a Wittig reagent to produce compound 6;
(3) reducing the compound 6 to give compound 7;
(4) allowing the compound 7 to react with 1,4-dioxane to produce compound 8 in an acidic condition;
(5) oxidizing the compound 8 to give compound 9;
(6) allowing the compound 9 to react with ethylene diamine to produce the compound of formula (I);
(7) allowing the compound of formula (I) to undergo a ring-opening reaction in an alkaline condition and thereby produce compound 11;
(8) allowing the compound 11 to react with trimethylchlorosilane and methanol in the presence of a tertially amine so as to produce compound 12;
(9) hydrolyzing the compound 12 in the presence of a quaternary ammonium compound to produce the compound of formula (II);
   wherein the compounds 5, 6, 7, 8, 9, 10, 11, and 12 respectively have the following structures,

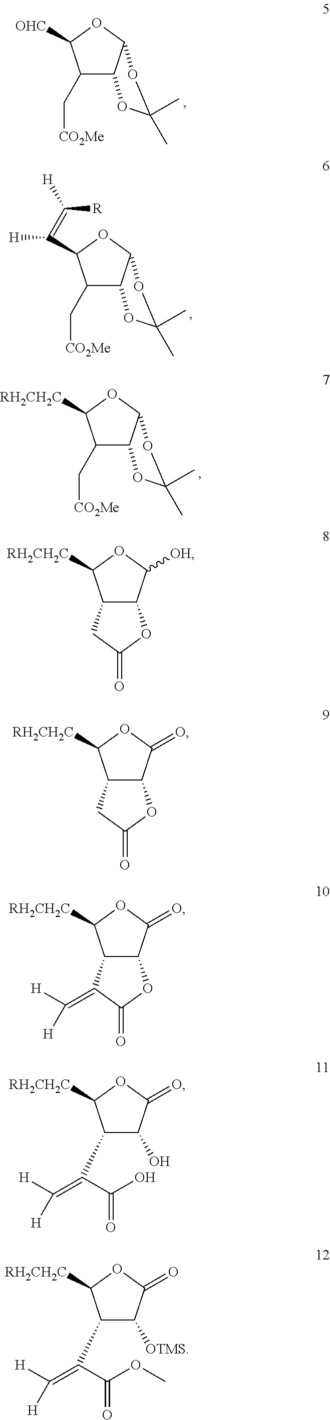

According to some embodiments, the step (1) comprises,
(a) let diacetone D-glucose react with pyridiunm dichromate to produce compound 2;
(b) let the compound 2 react with tri-ethyl phosphonoacetate to produce compound 3;
(c) allowing the compound 3 to undergo a ring-opening reaction to generate compound 4; and
(d) let the compound 4 react with an oxidizing agent and subsequently with a reducing agent to produce the compound 5;
   wherein, the compounds 2, 3, and 4 respectively have the following structures,

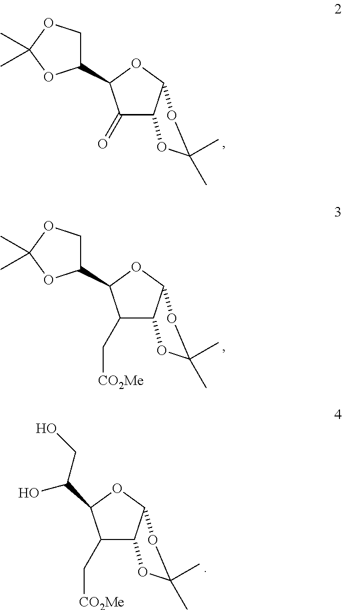

According to some embodiments, the Wittig reagent in the step (2) is C₄₋₁₂ alkyltriphenylphosphonium bromide or C₄₋₁₂ alkenylltriphenylphosphonium bromide. In one preferred example, the Wittig reagent is hexyltriphenylphosphonium bromide.

According to some embodiments, the step (6) comprises,
(e) let the compound 9 react with methyl methoxymagnesium carbonate in an inert environment; and
(f) allowing the product of the step (e) to react with an acid and subsequently with diethyl amine to produce the compound of formula (I).

According to some embodiments, the step (7) is performed in the presence of potassium hydroxide or sodium hydroxide.

According to some embodiments, the quaternary ammonium compound in the step (9) is tetrabutylammonium fluoride, tetrabutylammonium chloride, tetrabutylammonium bromide, tetrabutylammonium iodide, tetrabutylammonium perchlorate, tetrabutylammonium hexafluorophosphate, or tetrabutylammonium acetate.

The details of one or more embodiments of the invention are set forth in the accompanying description below. Other features and advantages of the invention will be apparent from the detail descriptions, and from claims.

### BRIEF DESCRIPTION OF THE DRAWINGS

The present description will be better understood from the following detailed description read in light of the accompanying drawings, where:

FIG. 1 are TEM photographs of MRSA (a-f) and A. baumannii (g-l) after being treated with the compounds of example 2 (128 μM) and fosfomycin (64 μM), in which (a) and (g) are un-treated S. aureus and A. baumannii; (b) and (g) are those treated with the compound 10d, while (c) and (i) are treated with the compound of 13d; (d) and (j) are treated with the compound 14; (e) and (k) are treated with the compound 15; (f) and (i) are treated with fosfomycin, in which CW, CM, IM, and OM respectively denote cell walls, cell membranes, inner membranes, and outer membranes; and

FIG. 2 illustrates the effect of the compounds of example 2 on the viability of macrophages.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. DEFINITIONS

For convenience, certain terms employed in the context of the present disclosure are collected here. Unless defined otherwise, all technical and scientific terms used herein have the same meaning as commonly understood by one of the ordinary skill in the art to which this invention belongs.

Unless otherwise indicated, the term “alkyl” means a straight chain, branched and/or cyclic (“cycloalkyl”) hydrocarbon having from 1 to 20 (e.g., 1 to 10, 1 to 9, 1 to 8, 1 to 7, 1 to 6, 1 to 5, 1 to 4, 1 to 3, 1 to 2, or 1) carbon atoms. Examples of alkyl groups include methyl, ethyl, propyl, isopropyl, n-butyl, t-butyl, isobutyl, 2-isopropyl-3-methyl butyl, pentyl, pentan-2-yl, hexyl, isohexyl, heptyl, heptan-2-yl, 4,4-dimethylpentyl, octyl, 2,2,4-trimethylpentyl, nonyl, decyl, undecyl and dodecyl. Cycloalkyl moieties may be monocyclic or multicyclic, and examples include cyclopropyl, cyclobutyl, cyclopentyl, and cyclohexyl. Unless otherwise specified, each instance of an alkyl group is independently optionally substituted, i.e., unsubstituted (an “unsubstituted alkyl”) or substituted (a “substituted alkyl”) with one or more substituents. In certain embodiments, the alkyl group is unsubstituted C₂₋₁₀ alkyl; preferably, unsubstituted C₂₋₆ alkyl; and more preferably, unsubstituted C₄₋₆ alkyl. In one preferred example, the alkyl group is n-hexyl.

Unless otherwise indicated, the term “alkenyl” means a straight chain, branched and/or cyclic hydrocarbon having from 2 to 20 (e.g., 2 to 10, 2 to 9, 2 to 8, 2 to 7, 2 to 6, 2 to 5, 2 to 4, 2 to 3, or 2) carbon atoms, and including one or more carbon-carbon double bond. Representative alkenyl moieties include vinyl, allyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, and 3-nonenyl. The one or more carbon-carbon double bonds can be internal (such as in 2-butenyl) or terminal (such as in 1-butenyl). Unless otherwise specified, each instance of an alkenyl group is independently optionally substituted, i.e., unsubstituted (an “unsubstituted alkenyl”) or substituted (a “substituted alkenyl”) with one or more substituents. In certain embodiments, the alkenyl group is substituted C₂₋₁₀ alkenyl; preferably, unsubstituted C₂₋₆ alkenyl; and more preferably, unsubstituted C₄₋₆ alkenyl.

Unless otherwise indicated, the term “substituted,” when used to describe a chemical structure or moiety, refers to a derivative of that structure or moiety wherein one or more of its hydrogen atoms is substituted with one or more of: alkoxy, alkyl, aryl, halo, haloalkyl, or hydroxyl.

The term “subject” or “patient” refers to an animal including the human species that is treatable with the method of the present invention. The term “subject” or “patient” intended to refer to both the male and female gender unless one gender is specifically indicated. Accordingly, the term “subject” or “patient” comprises any mammal which may benefit from the treatment method of the present disclosure.

The term “administered”, “administering” or “administration” are used interchangeably herein to refer a mode of delivery, including, without limitation, intravenously, intramuscularly, intraperitoneally, intraarterially, subcutaneously, or transdermally administering an agent (e.g., a compound or a composition) of the present invention.

The term “an effective amount” as used herein refers to an amount effective, at dosages, and for periods of time necessary, to achieve the desired result with respect to the treatment of a disease. For example, in the treatment of an infection, an agent (i.e., a compound or a composition) which decrease, prevents, delays or suppresses or arrests any symptoms of the infection would be effective. An effective amount of an agent is not required to cure a disease or condition but will provide a treatment for a disease or condition such that the onset of the disease or condition is delayed, hindered or prevented, or the disease or condition symptoms are ameliorated. The effective amount may be divided into one, two or more doses in a suitable form to be administered at one, two or more times throughout a designated time period.

The term “a sufficient amount” as used herein refers to an amount suffice at dosages, and for periods of time necessary, to achieve the desired result with respect to suppress the growth of gram-positive bacteria so that it is continuously present for a sufficient period of time to help suppress or inhibit the growth of gram-positive bacteria. In preferred examples, a sufficient amount of a compound of formula (I), (II), or a combination thereof is brought into contact with a gram-positive bacteria for a certain period of time such that the growth of gram-positive bacteria is suppressed for at least 80%, such as 80, 85, 90, 95, or 99%, as compared with that of the un-treated gram-positive bacteria.

The term “treatment” as used herein are intended to mean obtaining a desired pharmacological and/or physiologic effect, e.g., inhibiting the growth of gram-positive bacteria. The effect may be prophylactic in terms of completely or partially preventing a disease or symptom thereof and/or therapeutic in terms of a partial or complete cure for a disease and/or adverse effect attributable to the disease. “Treatment” as used herein includes preventative (e.g., prophylactic), curative or palliative treatment of a disease in a mammal, particularly human; and includes: (1) preventative (e.g., prophylactic), curative or palliative treatment of a disease or condition (e.g., an infection) from occurring in an individual who may be pre-disposed to the disease but has not yet been diagnosed as having it; (2) inhibiting a disease (e.g., by arresting its development); or (3) relieving a disease (e.g., reducing symptoms associated with the disease). According to specific embodiments of the present disclosure, an effective amount of a derivative of avenaciolide (i.e., the compound of formula (I) or (II)) is administered to a subject suffering from an infection caused by a gram-positive bacteria, so that the number of the gram-positive bacteria in the subject is reduced by at least 80%, such as 80, 85, 90, 95, or 99%, as compared with that of the un-treated subject, and thereby alleviate or ameliorate one or more symptoms associated with the disease, the severity of one or more symptoms associated with the disease and/or the progression of the disease. In preferred embodiments, an effective amount of the compound of formula (I) or (II) of the present disclosure is administered to a subject suffering from an infection associated with a disease (e.g., pneumonia, sepsis, cornea infection, skin infection, an infection in the central neuron system, or a toxic shock syndrome), so as to alleviate or ameliorate one or more symptoms associated with the disease, and thereby achieving the purpose of treating the disease.

Unless otherwise indicated, the term “gram-positive bacteria” as used herein intends to encompass aerobic gram-positive cocci, aerobic gram-positive rods, anaerobic gram-positive rods, and anaerobic gram-positive cocci. Examples of the aerobic gram-positive cocci include, but are not limited to, Staphylococcus agalactiae, Staphylococcus aureus, Staphylococcus epidermidis, Staphylococcus pneumonia, Staphylococcus pyrogens, Staphylococcus saprophyticus, Escherichia Coli, Enterococcim Streptococcus viridians, and Enterococcus faecalis. Examples of the aerobic gram-positive rods include, but are not limited to, Bacillus anthracis, Bacillus cereus, Bacillus subtilis, Biofidobacteria bifidum, Lactobacillius sp., Listeria monocytogens, Norcardia sp., Rhodococcus equi, Erysipelothrix rhusiopathiae, Corynebacterium diptheriae, and Propionbacterium acnes. Examples of the anaerobic gram-positive cocci includes, but is not limited to, Reptostreptoccus sp. Examples of the anaerobic gram-positive rods include, but are not limited to, Actinomyces sp., Clostridium botulinum Clostridium difficile, Clostridium perfringes, Clostridium tetani, and Clostridium scindens.

It should also be noted that if the stereochemistry of a structure or a portion of a structure is not indicated with, for example, bold or dashed lines, the structure or the portion of the structure is to be interpreted as encompassing all stereoisomers of it. Similarly, names of compounds having one or more chiral centers that do not specify the stereochemistry of those centers encompass pure stereoisomers and mixtures thereof. Moreover, any atom shown in a drawing with unsatisfied valences is assumed to be attached to enough hydrogen atoms to satisfy the valences. In addition, chemical bonds depicted with one solid line parallel to one dashed line encompass both single and double (e.g., aromatic) bonds, if valences permit.

The singular forms “a”, “and”, and “the” are used herein to include plural referents unless the context clearly dictates otherwise.

### 2. AVENACIOLIDE DERIVATIVES

The present disclosure is based, at least in part, unexpected discovery that the some avenaciolide derivatives, particularly the compound of formula (I) or (II), are capable of suppressing the growth of a gram-positive bacteria, including the drug-resistant gram-positive microorganisms; thus the avenaciolide derivatives of the present disclosure may be used as lead compounds for the development of a medicament for treating a disease associated with an infection of a gram-positive bacteria.

The avenaciolide derivatives are those having formula (I) or (II),

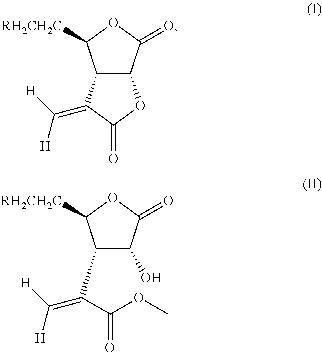

wherein R is C₂₋₁₀ alkyl or C₂₋₁₀ alkenyl.

According to certain embodiments, R of the compound of formula (I) is C₃₋₆ alkyl. In one example, R is C₃ alkyl, which includes, but is not limited to, n-propyl and isopropyl. In another example, R is C₄ alkyl, which includes, but is not limited to, n-butyl, sec-butyl and tert-butyl. In still another example, R is C₅ alkyl, which includes, but is not limited to, n-pentyl, isopentyl and neopentyl. In a further example, R is C₆ alkyl, which includes, but is not limited to, n-hexyl, 2-methyl-1-pentyl, 3-methyl-1-pentyl, 1,2-dimethyl-1-butyl, 2,2-dimethyl-1-butyl, 3,3-dimethyl-1-butyl, and 1,2,2-trimethyl-1-propyl.

According to other embodiments, R of the compound of formula (II) is C₃₋₆ alkyl. In one example, in the compound of formula (I), R is C₃ alkyl, which includes, but is not limited to, n-propyl and isopropyl. In another example, R is C₄ alkyl, which includes, but is not limited to, n-butyl, sec-butyl and tert-butyl. In still another example, R is C₅ alkyl, which includes, but is not limited to, n-pentyl, isopentyl and neopentyl. In a further example, R is C₆ alkyl, which includes, but is not limited to, n-hexyl, 2-methyl-1-pentyl, 3-methyl-1-pentyl, 1,2-dimethyl-1-butyl, 2,2-dimethyl-1-butyl, 3,3-dimethyl-1-butyl, and 1,2,2-trimethyl-1-propyl.

Preferably, in the compound of formula (I) or (II), R is n-hexyl, which respectively correspond to compound 10d of example 1.8 and compound 13d of example 1.11.

Each compound of formula (I) or (II) is a derivative of avenaciolide, and some of them may be synthesized in accordance with methods known in the related art. For example, compound 10d may be synthesized in accordance with the well-known methods, such as those described by Steven et al (J. Org Chem. (1992) 57, 2228-2235), Chen et al (J. Org Chem. (1999) 64, 8311-8318), and Santos et al (J. Org Chem. (2013) 78, 1519-1524).

Another approach of obtaining avenaciolide derivatives is by isolating the desired avenaciolide derivatives from the Neosartoya fischeri culture. For example, Yang et al described a process of isolating the desired avenaciolide derivatives (e.g., compound 13d of the present disclosure) from the Neosartoya fischeri culture by use of high performance liquid chromatography (see Plant Med (2010) 76:1701-1705).

Each compound of formula (I) or (II) comprises one or more asymmetric carbon, thereby give rise to various stereoisomers, including enantiomers, diastereomers, and a racemic mixture thereof. The present invention therefore encompasses at least, the enantiomers, the diastereomers of the compound of formula (I) or (II), a racemic mixture thereof, and/or a combination thereof. The enantiomers of the compound of formula (I) or (II) may be prepared by chiral synthesis or enantioselective synthesis, in which a specific chiral compound is used as the starting material for the synthesis of a desired stereocompound. Alternatively, the compound of formula (I) or (II) may be obtained by routine isolating techniques, which include, and are not limited to, crystallization, chromatography, and the use of resolving agents. For example, each enantiomers may be isolated from the racemic mixture by use of HPLC. Alternatively, one enantiomer is separated from the other enantiomer by allowing its racemic mixture to react with a resolving agent, which allows one enantiomer to become solvable in the resolving agent while the other enantiomer remains precipitated. The present invention also encompasses the structural isoforms of the compound of formula (I) or (II), such as those in cis- and/or trans-conformations, either with or without the presence of double bond(s) in its structure.

### 3. SYNTHESIS OF AVENACIOLIDE DERIVATIVES

The compound of formula (I) or (II)) of the present disclosure is synthesized by the method described bellowed, in which diacetone D-glucose is employed as the starting material.

In general, the present method includes steps of,
(1) using diacetone D-glucose as a starting material to produce compound 5;
(2) allowing the compound 5 to react with a Wittig reagent to produce compound 6;
(3) reducing the compound 6 to give compound 7;
(4) allowing the compound 7 to react with 1,4-dioxane to produce compound 8 in an acidic condition;
(5) oxidizing the compound 8 to give compound 9;
(6) allowing the compound 9 to react with ethylene diamine to produce the compound of formula (I);
(7) allowing the compound of formula (I) to undergo a ring-opening reaction in an alkaline condition so as to produce compound 11;
(8) allowing the compound 11 to react with trimethylchlorosilane and methanol in the presence of a tertially amine so as to produce compound 12;
(9) hydrolyzing the compound 12 in the presence of a quaternary ammonium compound to produce the compound of formula (II);
   wherein the compounds 5, 6, 7, 8, 9, 10, 11, and 12 respectively have the following structures,

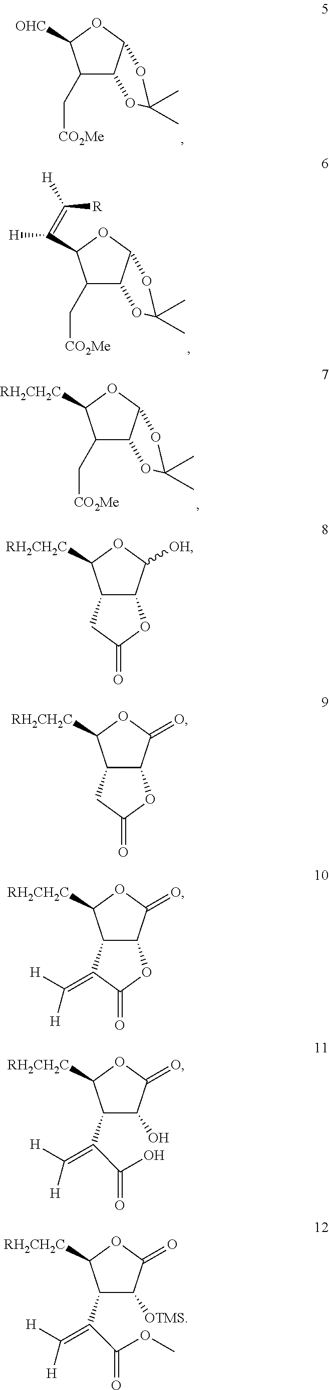

According to specific embodiments, the step (1) comprises,
(a) let diacetone D-glucose react with pyridium dichromate to produce compound 2;
(b) let the compound 2 react with tri-ethyl phosphonoacetate to produce compound 3;
(c) allowing the compound 3 to undergo a ring-opening reaction to generate compound 4; and
(d) let the compound 4 react with an oxidizing agent and subsequently with a reducing agent to produce the compound 5;
   wherein, the compounds 2, 3, and 4 respectively have the following structures,

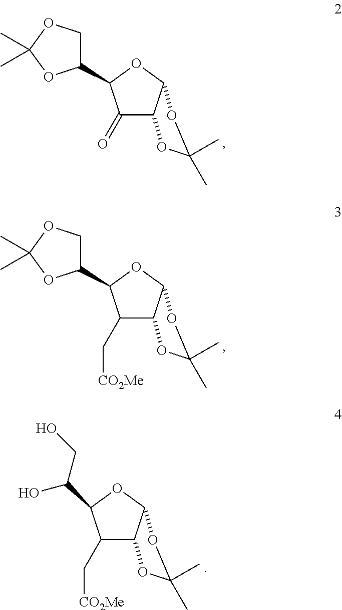

According to some embodiments, the oxidizing agent suitable for use in the step (d) may be the alkali metal salt of periodic acid, such as potassium periodate and sodium periodate. Examples of the reducing agent include, but are not limited to, the alkali metal salts of borohydride or aluminum hydride, such as sodium borohydride, and lithium aluminum hydride. In one example, the compound 4 in step (d) is allowed to react, sequentially, with sodium periodate (i.e., an oxidizing agent) and sodium borohydride (i.e., the reducing agent) so as to produce the compound 5.

According to some embodiments, the Wittig reagent in the step (2) is C₄₋₁₂ alkyltriphenylphosphonium bromide or C₄₋₁₂ alkenylltriphenylphosphonium bromide. In one preferred example, the Wittig reagent is hexyltriphenylphosphonium bromide.

According to some embodiments, in the step (3), the compound 6 is hydrogenated (or reduced) to the compound 7 in the present of a catalyst (e.g., 10% platinum/carbon).

According to some embodiments, in the step (5), a Jones oxidation is performed so as to convent the secondary hydroxyl group of the compound 8 to a ketone group and thereby generates the compound 9. The Jones oxidation is achieved by use of a Jones reagent, which is constituted by dissolving chromium trioxide in a solution of diluted sulphuric acid and acetone. According to some optional embodiments, chromium trioxide in the Jones reagent may be replaced by potassium dichromate.

According to some embodiments, the step (6) comprises,
(e) let the compound 9 react with methyl methoxymagnesium carbonate in an inert environment; and
(f) allowing the product of the step (e) to react, in sequence, with an acid and diethyl amine, to produce the compound of formula (I).

According to some embodiments, in the compound of formula (I), R is C₃₋₆ alkyl. In one example, R is C₃ alkyl, which includes, but is not limited to, n-propyl and isopropyl. In another example, R is C₄ alkyl, which includes, but is not limited to, n-butyl, sec-butyl and tert-butyl. In still another example, R is C₅ alkyl, which includes, but is not limited to, n-pentyl, isopentyl and neopentyl. In a further example, R is C₆ alkyl, which includes, but is not limited to, n-hexyl, 2-methyl-1-pentyl, 3-methyl-1-pentyl, 1,2-dimethyl-1-butyl, 2,2-dimethyl-1-butyl, 3,3-dimethyl-1-butyl, and 1,2,2-trimethyl-1-propyl. Preferably, in the compound of formula (I), R is n-hexyl, which corresponds to the compound 10d of the present disclosure.

Preferably, in the compound of formula (I) or (II), R is n-hexyl, which respectively correspond to compound 10d of example 1.8 and compound 13d of example 1.11.

According to some embodiments, the step (7) is performed in the presence of potassium hydroxide or sodium hydroxide.

According to some embodiments, the quaternary ammonium compound in the step (9) is any of tetrabutylammonium fluoride, tetrabutylammonium chloride, tetrabutylammonium bromide, tetrabutylammonium iodide, tetrabutylammonium perchlorate, tetrabutylammonium hexafluorophosphate, or tetrabutylammonium acetate. Preferably, the quaternary ammonium compound in the step (9) is tetrabutylammonium fluoride.

According to specific embodiments, in the compound of formula (II), R is C₃₋₆ alkyl. In one example, R is C₃ alkyl, which includes, but is not limited to, n-propyl and isopropyl. In another example, R is C₄ alkyl, which includes, but is not limited to, n-butyl, sec-butyl and tert-butyl. In still another example, R is C₅ alkyl, which includes, but is not limited to, n-pentyl, isopentyl and neopentyl. In a further example, R is C₆ alkyl, which includes, but is not limited to, n-hexyl, 2-methyl-1-pentyl, 3-methyl-1-pentyl, 1,2-dimethyl-1-butyl, 2,2-dimethyl-1-butyl, 3,3-dimethyl-1-butyl, and 1,2,2-trimethyl-1-propyl. Preferably, in the compound of formula (II), R is n-hexyl, which corresponds to the compound 13d of the present disclosure.

### 4. USES OF AVENACIOLIDE DERIVATIVES

Also within the scope of the present disclosure is a method for treating a subject suffering from a disease associated with an infection caused by a gram-positive bacteria. The method includes steps of, administering an effective amount of the compound of formula (I) or (II) to the subject, so as to alleviate or ameliorate one or more symptoms related to the disease.

Examples of the bacteria that may cause an infection of a subject include, but are not limited to, Bacillus anthracis, Bacillus subtilis, Bacillus cereus, Corynebacterium diphtheria, Clostridium tetani, Clostridium botulinum, Clostridium perfringes, Clostridium difficile, Clostridium scindens, Enterococcim Streptococcus viridians, Enterococcus faecalis, Erysipelothrix rhusiopathiae, Escherichia Coli, Listeria monocytogens, Propionbacterium acnes, Rhodococcus equi, Staphylococcus agalactiae, Staphylococcus aureus, Staphylococcus epidermidis, Staphylococcus pneumonia, Staphylococcus pyrogens, or Staphylococcus saprophyticus.

According to certain embodiment, the subject has skin abscess, furuncle or skin boil, and thereby resulting the subject prone to bacterial infection. Examples of the disease associated with an infection caused by a gram-positive bacteria include, but are not limited to, pneumonia, sepsis, cornea infection, skin infection, an infection in the central neuron system, and a toxic shock syndrome.

Take sepsis as an example, the most common gram-positive bacteria responsible for causing sepsis is Staphylococcus aureus and/or Streptococcus pneumonia; however, any of the afore-identified gram-positive bacteria may cause sepsis in a subject. Accordingly, certain embodiments of the present disclosure are directed to a method of treating a subject having sepsis caused by the infection of Staphylococcus aureus or Streptococcus pneumonia. According to certain examples, the Staphylococcus aureus is a drug-resistant Staphylococcus aureus, such as methicillin-resistant Staphylococcus aureus (MRSA).

According to further embodiments, the present invention is directed to a method for treating s subject suffering from a disease associated with an infaction caused by Enterococcus faecalis. In some examples, the Enterococcus faecalis is a vancomycin resistant Enterococcus faecalis.

In general, the compound of formula (I) or (II) is administered to the subject in need thereof in an amount of about 1-100 mg/Kg body weight, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 mg/Kg body weight; preferably about 20-80 mg/Kg body weight, such as 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, and 80 mg/Kg body weight; more preferably about 40-60 mg/Kg body weight, such as 40, 41, 42, 43, 44, 45, 46, 47, 48, 49, 50, 51, 52, 53, 54, 55, 56, 57, 58, 59, and 60 mg/Kg body weight. The amount may be administered in a single dosage or in multiple dosages in a day, such as in 2, 3, or 4 dosages per day. The actual amount of the compound of formula (I) or (II) will depend on the specific symptoms of the subject, and the physical conditions of the subject such as age, sex, medical history and etc.; and may be readily determined by the attending physician in accordance with his/her experience.

In some embodiments, the method further includes administering another antibiotic and/or antibacterial agent before, concurrently with, or after the administration of the compound of formula (I) or (II). Examples of the antibiotic that may be used with the present composition include, but are not limited to, acumycin, ampicillin, amoxycillin, amphotericins, antimycins, anglomycin, avermectins, azithromycin, boromycin, carbomycins, carbapenem, ceftazidime, cethromycin, chloramphenicol, chalcomycin, ciprofloxacin, concanamycins, cirramycin, clarithromycin, colistin, cycloxacillin, daptomycin, desmethyl azithromycin, desertomycins, dihydropikromycin, dirithromycin, doxycycline, enramycin, erythromycin, flurithromycin, flumequin gentamycin, juvenimicins, kujimycins, lankamycins, lincomycin, litorin, leucomycins, megalomicins, meropenem, methymycin, midecamycins, mycinamicin I, mycinamicin II, mycinamicin III, mycinamicin IV, mycinamicin V, mycinamicin VI, mycinamicin VII, mycinamicin VIII, narbomycin, neoantimycin, neomethymycin, netilmicin, neutromycin, niddamycins, norfioxacin, oleandomycins, oligomycins, ossamycin, oxacillin, oxolinic acid, penicillin, pikromycin, piperacillin, platenomycins, rapamycins, relomycin, rifamycins, rosaramicin, roxithromycin, virginiamycin, spiramycin, sporeamycin, staphococcomycin, streptomycin, sulfamethoxazole, swalpamycin, telithromycin, teicoplanin, timentin, tobramycin, ticarcillin, trimethoprim, tetracyclin, zlocillin, and/or a combination thereof.

### 5. PHARMACEUTICAL COMPOSITION

The present disclosure also encompasses a pharmaceutical composition for treating a disease associated with an infection caused by a gram-positive bacteria, or for suppressing the growth of a gram-positive bacteria. The pharmaceutical composition comprises an effective amount of the compound of formula (I) or (II); and a pharmaceutically acceptable excipient.

The compound of formula (I) or (II) of this invention is present at a level of about 0.1% to 99% by weight, based on the total weight of the pharmaceutical composition. In some embodiments, the compound of formula (I) or (II) of this invention is present at a level of at least 1% by weight, based on the total weight of the pharmaceutical composition. In certain embodiments, the compound of formula (I) or (II) of this invention is present at a level of at least 5% by weight, based on the total weight of the pharmaceutical composition. In still other embodiments, the compound of formula (I) or (II) of this invention is present at a level of at least 10% by weight, based on the total weight of the pharmaceutical composition. In still yet other embodiments, the compound of formula (I) or (II) of this invention is present at a level of at least 25% by weight, based on the total weight of the pharmaceutical composition.

The pharmaceutical composition is prepared in accordance with acceptable pharmaceutical procedures, such as described in Remington's Pharmaceutical Sciences, 17^{th} edition, ed. Alfonoso R. Gennaro, Mack Publishing Company, Easton, Pa. (1985). Pharmaceutically acceptable excipients are those that are compatible with other ingredients in the formulation and biologically acceptable.

According to some optional embodiments, the pharmaceutical composition further includes, an antibiotic. Examples of suitable antibiotic to be used in the present pharmaceutical composition include, but are not limited to, acumycin, ampicillin, amoxycillin, amphotericins, antimycins, anglomycin, avermectins, azithromycin, boromycin, carbomycins, carbapenem, ceftazidime, cethromycin, chloramphenicol, chalcomycin, ciprofloxacin, concanamycins, cirramycin, clarithromycin, colistin, cycloxacillin, daptomycin, desmethyl azithromycin, desertomycins, dihydropikromycin, dirithromycin, doxycycline, enramycin, erythromycin, flurithromycin, flumequin gentamycin, juvenimicins, kujimycins, lankamycins, lincomycin, litorin, leucomycins, megalomicins, meropenem, methymycin, midecamycins, mycinamicin I, mycinamicin II, mycinamicin III, mycinamicin IV, mycinamicin V, mycinamicin VI, mycinamicin VII, mycinamicin VIII, narbomycin, neoantimycin, neomethymycin, netilmicin, neutromycin, niddamycins, norfloxacin, oleandomycins, oligomycins, ossamycin, oxacillin, oxolinic acid, penicillin, pikromycin, piperacillin, platenomycins, rapamycins, relomycin, rifamycins, rosaramicin, roxithromycin, virginiamycin, spiramycin, sporeamycin, staphococcomycin, streptomycin, sulfamethoxazole, swalpamycin, telithromycin, teicoplanin, timentin, tobramycin, ticarcillin, trimethoprim, tetracyclin, zlocillin, and/or a combination thereof.

The compound of formula (I) or (II) of the present invention may be formulated into a single dosage suitable for oral, transmembrane (such as intranasal, sublingual, intravaginal, buccal, and/or endorectal), and/or parenteral administration (e.g., subcutaneous, intravenous, intramuscular, intraperitoneal or bolus injection) Examples of the dosage include, but are not limited to, tablets, caplets, capsules (e.g., soft elastic gelatin capsules), cachets, troches, lozengesm dispersions, suppositories, ointments, cataplasms (or poultices) creams, plasters, solutions, patches, aerosols, or gels. The compound of formula (I) or (II) of the present invention may be formulated into a liquid pharmaceutical compositions, which are sterile solutions, suspensions (e.g., water solvable or insolvable liquid suspension, oil-in-water emulsion or water-in-oil emulsion) or elixirs that can be administered by, for example, oral ingestion, or intravenous, intramuscular, subcutaneous or intraperitoneal injection.

The compound of the present invention is formulated in accordance with the intended routes for its administration. For example, if the compound of the present invention is intended to be administered by oral ingestion, an enteric coating may be applied on the formulation so as to prevent the compound of the present invention from being degraded in the acidic environment or until it reaches the intestines of the subject. The formulation may further include additional components that help deliver the compound of the present invention to its intended target site. In some examples, the compound of the present invention is enclosed in a liposome to prevent it from enzymatic degradation, and to help transporting the compound of the present disclosure through the circulation system of the subject, and/or across cell membrane to its intended cellular target site.

Further, the least soluble compound of the present invention may be formulated with additional agents, such as a solvating agent, an emulsifying agent and/or a surfactant, into a liquid formulation. Examples of the additional agent include, but are not limited to, cyclodextrin (e.g., α-cyclodextrin and β-cyclodextrin), and non-aqueous solvents, which include but are not limited to, ethanol, isopropanol, ethyl carbonate, ethyl acetate, benzyl alcohol, benzyl benzoate, propyl glycol, 1,3-butyl glycol, dimethyl formamide, dimethyl sulfoxide, biocompatible oils (e.g., cottonseed oil, peanut oil, corn oil, wheat germ oil, castor oil, olive oil, sesame oil, glycerol, tetrahydrogen furan, polyethyl glycol, fatty acid esters of sorbitan, and a combination thereof).

The amount of the compound of the present disclosure in the formulation varies with the route of administration. For example, formulations for acute treatment will contain larger amounts of one or more of the active compounds, as compared to formulations that are for chronic treatment. Similarly, parental formulations will comprise less amounts of one or more of the active compounds, as compared to formulations that are for oral ingestion. Also within the scope of the present disclosure are formulations suitable for other administration routes.

5.1 Formulation for Oral Ingestion

The compound of present disclosure may be formulation into compositions suitable for oral ingestion. Examples of such formulations include, but are not limited to, chewable tablets, tablets, capsules, and syrups, which may be prepared in accordance with procedures described in Remington's Pharmaceutical Sciences (18^{th} ed., Mack Publishing, Easton, Pa. (1990)). The oral formulation is prepared by mixing a pre-determined amount of the active compound and one or more pharmaceutically acceptable excipients in accordance with procedures well known in the related art.

Tablets and capsules are two most common forms of oral formulation, which may be either liquid or solid composition form. In general, the tablets and capsules are manufactured by mixing the active components with liquid or milled solid excipients, then press into pre-determined forms. The solid formulation may further include disintegrants, which increase solubility; and lubricants.

5.2 Formulation for Parental Administration

Parental formulations are those suitable for subcutaneous, intravenous (which includes bolus injection), intramuscular, and intraperitoneal injection. To this purpose, sterile injectable or suspension are required so as to prevent the recipients from microorganism infections. Suitable diluents or solvent for manufacturing sterile injectable solution or suspension include, but are not limited to, 1,3-butanediol, mannitol, water, Ringer's solution, and isotonic sodium chloride solution. Fatty acids, such as oleic acid and its glyceride derivatives are also useful for preparing injectables, as are natural pharmaceutically-acceptable oils, such as olive oil or castor oil. These oil solutions or suspensions may also contain alcohol diluent or carboxymethyl cellulose or similar dispersing agents. Other commonly used surfactants such as Tweens or Spans or other similar emulsifying agents or bioavailability enhancers that are commonly used in manufacturing pharmaceutically acceptable dosage forms can also be used for the purpose of formulation.

5.3 Transmembrane Formulation

Transmembrane formulations are those suitable for topical and tansmucosal uses, which include but are not limited to, ophthalmic solutions, sprays, aerosols, creams, lotions, ointments, gels, solutions, suspensions, skin patches and the like. The patches include reservoir type and matrix type skin patches, and may adhere onto the skin for a certain period of time to allow the active component to be adsorbed into the subject's body.

For topical administration, a wide variety of dermatologically acceptable inert excipients well known to the art may be employed. Typical inert excipients may be, for example, water, ethyl alcohol, polyvinyl pyrrolidone, propylene glycol, mineral oil, stearyl alcohol and gel-producing substances. All of the above dosages forms and excipients are well known to the pharmaceutical art. The choice of the dosage form is not critical to the efficacy of the composition described herein.

For transmucosal administration, the pharmaceutical compositions of this invention may also be formulated in a variety of dosage forms for mucosal application, such as buccal and/or sublingual drug dosage units for drug delivery through oral mucosal membranes. A wide variety of biodegradable polymeric excipients may be used that are pharmaceutically acceptable, provide both a suitable degree of adhesion and the desired drug release profile, and are compatible with the active agents to be administered and any other components that may be present in the buccal and/or sublingual drug dosage units. Generally, the polymeric excipient comprises hydrophilic polymers that adhere to the wet surface of the oral mucosa. Examples of polymeric excipients include, but are not limited to, acrylic acid polymers and copolymers; hydrolyzed polyvinylalcohol; polyethylene oxides; polyacrylates; vinyl polymers and copolymers; polyvinylpyrrolidone; dextran; guar gum; pectins; starches; and cellulosic polymers.

The present invention will now be described more specifically with reference to the following embodiments, which are provided for the purpose of demonstration rather than limitation.

### Examples

### Materials and Methods

Culture of Microorganisms

Various strains of microorganisms were used in the present disclosure, including Acinetobacter baumannii (ATCO 17978), Staphylococcus aureus (ATCO 29213), methicillin-resistant Staphylococcus aureus (MRSA) (ATCO 33592), Bacillus subtilis (ATCO 23857), and Escherichia Coli (ATCO 25922), which were purchased from American Type Culture Collection (ATCO), and cultured in Mueller-Hinton (MH) II agar plates or in MH broth.

Cell Lines and Culture

Mice macrophage cell line RAW 264.2 was used in the present disclosure. RAW 264.2 cells were cultured in RPMI 1640 medium supplemented with 10% fetal calf serum (FCS) and maintained in an environment of 5% CO₂/95% air at 37° C.

Minimum Inhibition Concentration (MIC) Assay

MIC assay is a standardized assay performed in accordance with the guidelines issued by Clinical and Laboratory Standard Institute (CLSI) that measures the ability of certain agent and/or a surface in inhibiting the growth of a microorganism or killing the microorganism after being in contact with that microorganism for 24 hrs. Accordingly, MIC is the lowest concentration of an anti-bacterial agent and/or a surface that will inhibit the visible growth of a microorganism after overnight culture.

In practice, microorganisms grown on the agar plates were re-suspended in phosphate-buffered saline (PBS) to an OD 600 of 0.1. Suitable amount of this suspension was added to the appropriate broth media at a final concentration of 5×10⁵ colony-forming units (CFU)/mL. Then, 98 μL of of the broth+cell solution for each strain was added to each well of 96-well assay plate. Solutions of the present compounds were freshly prepared in DMSO and then serial diluted in 2-fold steps in DMSO, resulting in a final concentrations of 0.004-256 μg/mL when diluted 1:50 in test broth. The initial inoculum for each strain was serially diluted in PBS and plated on appropriate agar plate media to ensure that the assay contained the desired number of CFU. MIC assay plates were incubated overnight at 37° C. under aerobic conditions.

AlamaBlue Cell Viability Assay

The alamarBlue cell viability assay uses a non-toxic, water-soluble resazurin dye that yields a fluorescent signal and a colorimetric change when incubated with metabolically active cells. The assay is based on the findings that metabolically active cells (or viable cells) may keep the culture medium in a reduced state, whereas the non-viable cells will turn the culture medium into an oxidized state, thereby rendering the redox indicator in the medium (i.e., resazurin dye) to convert from its non-fluorescent oxidized form (blue color) to its fluorescent reduced form (pink color). Thus, the cell viability may be monitored by the fluorescent signal changes at 590 nm.

Cells were plated in 96-well plates at a density of 5,000 cells/100 μL/well for 24 hrs, then 10 μL AlamarBlue™ reagent (AbD Serotee Ltd., Oxford, UK) and various concentrations (i.e., 12.5, 25, 50 or 100 μM) of the test compound (e.g., the compound of example 2) were added, and incubated for 24 hrs. The plates were then placed at a fluorescence reader and fluorescent signals at 590 nm were measured.

Transmission Electron Microscopy (TEM)

Exponential phase bacteria were treated with compounds at 4×MIC for 1 hr at 37° C. Then, the cells were washed and fixed and cut into thin slices and photographed using a Thin-layer TEM.

MuraA Assay

In a standardized assay, MuraA enzymes were pre-incubated with the substrate UNAG and an inhibitor for 10 min at 37° C. To determine the influence of UNAG on the binding process, experiments were performed in the absence of UNAG during pre-incubation period. The reaction was initiated by the addition of the second substrate PEP, resulting in a total volume of 1004 with the following concentrations: E. Coli MurA or MRSA MurA 25 nM, UNAG 310 μM, PEP 620 μM, 50 mM HEPES, pH 7.6, DMSO 1% (v/v). The reaction was stopped after 60 min at 37° C. by adding 100 μL of Lanzetta reagent containing malachite green solution (0.045% (w/v)) and ammonium heptamolybdate (8.4% (v/v)) at a ratio of 3:1 and with 0.03% (w/v) Tergitol NP-40 as dye-stabilizing detergent. After 10 min, the absorbance at 620 nm was measured using a FlexStation 3 Microplate Reader (Molecular Devices). Finally, dose-response curves were generated by measuring the enzyme activity of five replicates at least 8 different compound concentrations. The resulting data were plotted and IC₅₀ values indicating the concentration of the compound with a residual activity of 50% were determined.

Statistical Analysis

All data are presented as mean±standard deviation unless otherwise indicated. For multiple comparisons, analysis of variance (ANOVA) with Bonferroni adjustment was performed. A probability value of P<0.05 was considered to represent statistical significance.

### Example 1

### Synthesis of the Compound of Formula (I) and (II)

The compound of formula (I) or (II) were synthesized in accordance with procedures described in the following examples, and their structures were respectively confirmed by NMR spectra.

1.1 Compound 2

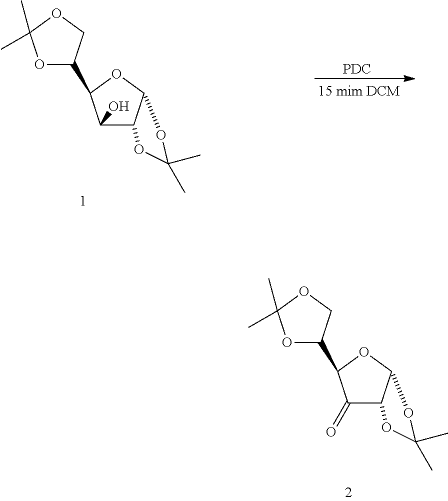

To 1 mmole of the compound 1 and 1.5 mmole PDC (Aldrich) in 5 ml CH₂Cl₂, 800 mg freshly activated molecular sieve powder 6 (3 Å, Aldrich) was added, followed by the addition of 100 μl anhydrous AcOH, the mixture was then stirred magnetically at room temperature. The reaction was carefully followed by thin layer chromatography and worked up in the following way. The reaction mixture was stirred with celite (ca. 500 mg/mmole) for about 20 min., filtered and evaporated under reduced pressure with toluene to remove pyridine and/or AcOH. The resulting dark brown residue was treated with a hydrocarbon solvent or diethyl ether, filtered through anhydrous, powdered MgSO₄ and evaporated to give pure product. For carbonyl products insoluble in these solvents, the residue was taken up in an anhydrous, aprotic solvent like ethyl acetate, filtered through silica gel and evaporated to obtain pure products of compound 2 (yield 70%).

¹H NMR (500 MHz, CDCl₃) δ5.47 (1H, s), 5.08 (1H, s), 4.44 (1H, m), 4.02 (1H, d, J=5), 3.98 (2H, d, J=7), 1.27 (12H, s).

¹³C NMR (125 MHz, CDCl₃) δ 203 (C═O), 119.2 (C), 116.5 (C), 107.7 (CH), 80.5 (CH), 77.9 (CH), 68.2 (CH), 64.8 (CH2), 26.2 (4 CH3).

1.2 Compound 3

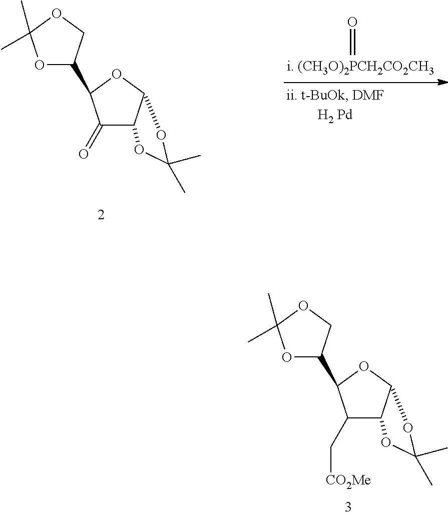

An ice-cold solution of phosphonoacetic acid trimethyl ester (11 ml) and potassium t-butoxide (2.5 g) in anhydrous N,N-dimethylformamide (11 ml) was added slowly to a solution (kept at 0° C.) of 5.5 g of compound 2 in 33 ml of anhydrous N,N-dimethylformamide. The reaction mixture was kept at 0° C. for 1 hr and then at a room temperature for 48 hr (or until all of compound was consumed as evidenced by monitoring by TLC on silica gel G using benzene-methanol (95:5) as developer). The solvent was removed under reduced pressure and the residue, after addition of 150 ml water, was extracted twice with ether (150 ml), the ether layer was washed with water (30 ml), dried over magnesium sulfate, and filtered, and the filtrate was evaporated under reduced pressure. This product consisted of a mixture of cis- and trans-unsaturated branched-chain sugars.

Preparative TLC of part of this product (0.35 g) using silica gel G and benzenemethanol (95:5) as developer gave two different unsaturated sugars (0.29 g) (in the ratio of 1:3 and each fraction having traces of contamination of the other): The mixture (4.7 g) of unsaturated sugars in 140 ml of ethanol was hydrogenated using 10% palladium on charcoal (2.2 g) as catalyst; 380 ml (1 mol equiv) of gas was absorbed. The catalyst was removed by filtration and the filtrate was then evaporated to give compound 3 (4 g, 85% yield) (homogeneous by chromatography) that was recrystallized from petroleum ether (bp 35-65° C.) or from methanol-water (4:1): mp 57-58° C.; [α]²²D +65° (c2, ethanol).

¹H NMR (500 MHz, CDCl₃) δ 5.24 (1H, d, J=6.25), 4.21 (1H, m), 4.20 (1H, m), 4.05 (1H, m), 3.98 (2H, d, J=4), 3.68 (OCH₃, s), 2.79 (1H, m), 2.27 (2H, d J=3.8), 1.27 (12H, s).

¹³C NMR (125 MHz, CDCl3) δ 173.1 (C═O), 121.9 (C), 119.5 (C), 110.4 (CH), 88.8 (CH), 78.7 (CH), 73.4 (CH), 67.0 (CH₂), 51.9 (CH₃), 32.8 (CH₂), 26.5 (2 CH₃), 26.2 (2 CH₃), 25.2 (CH).

1.3 Compound 4

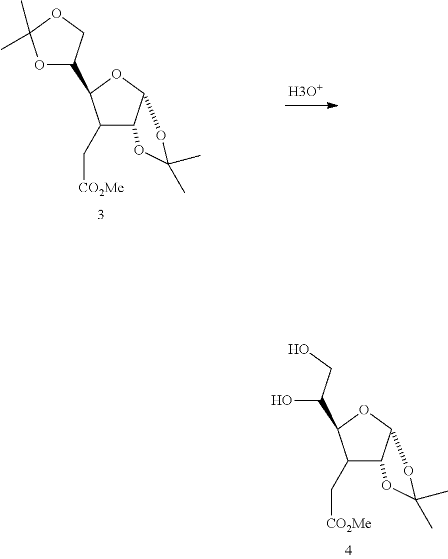

Partial hydrolysis of compound 3 to yield compound 4. To a solution of the branched-chain compound 3 (0.78 g (0.0027 mol)) in 4.5 ml of methanol, 4.5 ml of 0.8% sulfuric acid was added. The reaction mixture was left stand at room temperature for 3 hr, then neutralized with barium carbonate, boiled for a few minutes to coagulate the precipitate, and filtered. The filtrate was evaporated to dryness. 20 ml water and 4 mL chloroform were added to the residue, and the mixture was vigorously shaken. The chloroform extract was dried over anhydrous sodium sulfate, filtered, and evaporated to dryness to yield 0.04 g of starting material. The aqueous solution was evaporated to dryness and the residue was azeotroped with ethanol.

The resulting oil was extracted with boiling chloroform, dried with magnesium sulfate, filtered, and evaporated to dryness under reduced pressure: yield 0.622 g of oil (92%).

¹H NMR (500 MHz, CDCl₃) δ 5.24 (1H, d, J=6.25), 4.20 (1H, dd, J=2.2, 1.2), 3.81 (1H, dd), 3.81 (2H, s), 3.68 (3H, s), 3.65 (1H, s), 3.62 (1H, m), 3.58 (1H, s), 2.79 (1H, m), 2.27 (2H, d, J=), 1.27 (6H, s).

¹³C NMR (125 MHz, CDCl₃) δ 173.1 (C═O), 121.9 (C), 110.4 (CH), 88.8 (CH), 75.3 (CH), 70.6 (CH), 64.7 (CH2), 51.9 (CH3), 32.8 (CH2), 26.5 (CH3), 24.9 (CH).

1.4 Compound 5

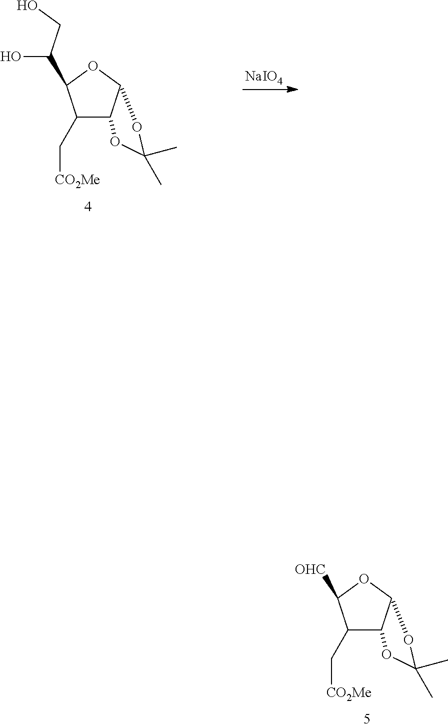

To a solution of the compound 4 (0.005 g) in 1.5 ml water and 0.3 ml ethanol, a solution of sodium metaperiodate (0.0045 g) in 0.25 ml water, was added. The reaction mixture was left stand at room temperature for 0.5 hr. Sodium borohydride (0.020 g) was added to the reaction mixture which was then left stand at room temperature for 35 min. Acetic acid (10%) (0.25 ml) was added to decompose excess sodium borohydride. The reaction mixture was evaporated under reduced pressure and the residue was azeotroped three times with ethanol. The product was separated by paper chromatography using water as developer to give 0.005 g of a nucleoside having an identical NMR spectrum with that of the compound 5.

¹H NMR (500 MHz, CDCl₃) δ 9.72 (1H, s), 5.24 (1H, d, J=3.8), 4.60 (1H, dd, J=2.1, 1.8), 4.20 (1H, s), 3.68 (3H, s), 3.02 (1H, m), 2.27 (2H, d, J=), 1.27 (6H, s).

¹³C NMR (125 MHz, CDCl₃) δ 200.8 (C═O), 173.1 (C═O), 121.9 (C), 112.5 (CH), 88.0 (CH), 85.8 (CH), 51.9 (CH₃), 26.8 (CH₂), 26.5 (CH₃), 23.3 (CH)

1.5 Compounds 6a, 6b, 6c, and 6d

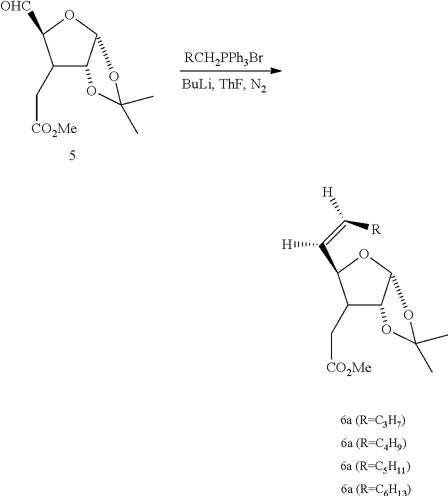

The alkyltriphenylphosphonium bromides were prepared by refluxing a solution of triphenylphosphine and the alkyl bromides (1:1) in toluene, under nitrogen atmosphere for 10 hours. The solids were separated by filtration, washed with dry diethyl ether and dried under reduced pressure (70 to 80% yields). The phosphonium salts thus obtained (0.018 mol) were dissolved in dry THF (37 mL), butyl lithium (0.011 mol) was added and the reaction mixtures were stirred for 0.5 h under a nitrogen atmosphere. A solution of the aldehyde (0.009 mol) in dry THF (5.0 mL) was added in drop wise manner and the stirring continued for 3 h at room temperature. Elimination of the THF, addition of water and extraction with Et₂O gave the crude reaction product. Purification (removal of phosphorus containing residue) was effected by chromatography on a silica gel column (hexane:EtOAc 3:1).

Compound 6a

¹H NMR (500 MHz, CDCl₃) δ5.68 (1H, d, ³J=4.1), 5.61 (1H, dt, J=10.9, 7.5), 5.28 (1H, ddt, J=10.9, 9.7, and 4.77 (1H, t, J=4.1), 4.53 (t, J=9.7), 3.69 (3H, s, OCH₃), 2.63 (2H, dd, J=16.7, 10.5), 2.27 (1H, dd, J=16.7, 4.1), 1.96 (2H, m), 1.44 (2H, m), 1.27 (6H, s), 0.9 (3H, s).

¹³C NMR (125 MHz, CDCl₃) δ 172.54 (C═O), 136.41 (═CH), 126.45 (═CH), 111.44 (CH), 104.97 (C), 80.57 (CH), 51.75 (OCH3), 46.02 (CH), 33.94 (CH₂), 28.90 (2CH₃), 26.62 (CH), 17.28 (CH₂), 15.10 (CH₂), 13.79 (CH₃)

Compound 6b

¹H NMR (500 MHz, CDCl₃) δ5.68 (1H, d, ³J=4.1), 5.61 (1H, dt, J=10.9, 7.5), 5.28 (1H, ddt, J=10.9, 9.7, and 4.77 (1H, t, J=4.1), 4.53 (t, J=9.7), 3.69 (3H, s, OCH₃), 2.63 (2H, dd, J=16.7, 10.5), 2.27 (1H, dd, J=16.7, 4.1), 1.96 (2H, m), 1.44 (2H, m), 1.29 (2H, m), 1.27 (6H, s), 0.9 (3H, s).

¹³C NMR (125 MHz, CDCl₃) δ 172.54 (C═O), 136.41 (═CH), 126.45 (═CH), 111.44 (CH), 104.97 (C), 80.57 (CH), 51.75 (OCH3), 46.02 (CH), 33.94 (CH₂), 28.90 (2CH₃), 26.62 (CH), 22.1 (CH₂), 17.28 (CH₂), 15.10 (CH₂), 13.79 (CH₃)

Compound 6c

¹H NMR (500 MHz, CDCl₃) δ5.68 (1H, d, ³J=4.1), 5.61 (1H, dt, J=10.9, 7.5), 5.28 (1H, ddt, J=10.9, 9.7, and 4.77 (1H, t, J=4.1), 4.53 (t, J=9.7), 3.69 (3H, s, OCH₃), 2.63 (2H, dd, J=16.7, 10.5), 2.27 (1H, dd, J=16.7, 4.1), 1.96 (2H, m), 1.44 (2H, m), 1.29 (4H, m), 1.27 (6H, s), 0.9 (3H, s).

¹³C NMR (125 MHz, CDCl₃) δ 172.54 (C═O), 136.41 (═CH), 126.45 (═CH), 111.44 (CH), 104.97 (C), 80.57 (CH), 51.75 (OCH₃), 46.02 (CH), 33.94 (CH₂), 28.90 (2CH₃), 26.62 (CH), 22.4 (CH₂), 22.1 (CH₂), 17.28 (CH₂), 15.10 (CH₂), 13.79 (CH₃)

Compound 6d

¹H NMR (500 MHz, CDCl₃) δ5.68 (1H, d, ³J=4.1), 5.61 (1H, dt, J=10.9, 7.5), 5.28 (1H, ddt, J=10.9, 9.7, and 4.77 (1H, t, J=4.1), 4.53 (t, J=9.7), 3.69 (3H, s, OCH₃), 2.63 (2H, dd, J=16.7, 10.5), 2.27 (1H, dd, J=16.7, 4.1), 1.96 (2H, m), 1.44 (2H, m), 1.29 (6H, broad), 1.27 (6H, s), 0.9 (3H, s).

¹³C NMR (125 MHz, CDCl₃) δ 172.54 (C═O), 136.41 (═CH), 126.45 (═CH), 111.44 (CH), 104.97 (C), 80.57 (CH), 51.75 (OCH₃), 46.02 (CH), 33.94 (CH₂), 28.90 (2CH₃), 26.62 (CH), 22.4 (CH₂), 22.28 (CH₂), 22.1 (CH₂), 17.28 (CH₂), 15.10 (CH₂), 13.79 (CH₃)

1.6 Compounds 7a, 7b, 7c, and 7d

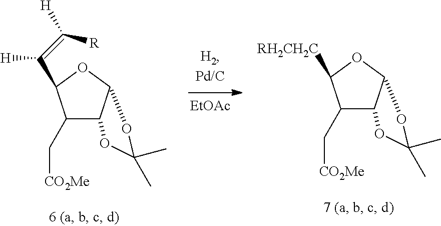

To 0.035 mol L⁻¹ solutions of the compounds 6 (a, b, c, d) in ethyl acetate (100 mL) 50 mg of Pd/C (10%) were added. The suspensions were stirred for 16 h at room temperature under hydrogen atmosphere. The mixtures were filtered and the filtrates were evaporated.

Compound 7a

¹H NMR (500 MHz, CDCl₃) δ5.24 (1H, d, ³J=3.8), 4.2 (1H, dd, J=4.5, 3.8), 3.8 (1H, ddd, J=10.2, 7.9, 2.4), and 3.69 (3H, s, OCH₃), 2.66 (H, dd, J=16.9, 10.2), 2.32 (1H, dd, J=16.9, 4.5), 2.04 (1H, tt, J=10.2, 4.5), 1.42 (2H, m), 1.31 (2H, m), 1.27 (6H, m), 1.25 (4H, m), 0.9 (3H, s).

¹³C NMR (125 MHz, CDCl₃) δ 173.1 (C═O), 121.9 (C), 110.1 (CH), 88.5 (CH), 83.0 (CH), 51.75 (OCH₃), 33.1 (CH₂), 32.5 (CH₂), 32.1 (CH₂), 31.1 (CH), 26.5 (2CH₃), 25.7 (CH₂), 22.7 (CH₂), 14.1 (CH₃)

Compound 7b

¹H NMR (500 MHz, CDCl₃) δ5.24 (1H, d, ³J=3.8), 4.2 (1H, dd, J=4.5, 3.8), 3.8 (1H, ddd, J=10.2, 7.9, 2.4), and 3.69 (3H, s, OCH₃), 2.66 (H, dd, J=16.9, 10.2), 2.32 (1H, dd, J=16.9, 4.5), 2.04 (1H, tt, J=10.2, 4.5), 1.42 (2H, m), 1.31 (2H, m), 1.29 (2H, m), 1.27 (6H, m), 1.25 (4H, m), 0.9 (3H, s).

¹³C NMR (125 MHz, CDCl₃) δ 173.1 (C═O), 121.9 (C), 110.1 (CH), 88.5 (CH), 83.0 (CH), 51.75 (OCH₃), 33.1 (CH₂), 32.5 (CH₂), 32.1 (CH₂), 31.1 (CH), 26.5 (2CH₃), 25.7 (CH₂), 23.7 (CH₂), 22.7 (CH₂), 14.1 (CH₃).

Compound 7c

¹H NMR (500 MHz, CDCl₃) δ5.24 (1H, d, ³J=3.8), 4.2 (1H, dd, J=4.5, 3.8), 3.8 (1H, ddd, J=10.2, 7.9, 2.4), and 3.69 (3H, s, OCH₃), 2.66 (H, dd, J=16.9, 10.2), 2.32 (1H, dd, J=16.9, 4.5), 2.04 (1H, tt, J=10.2, 4.5), 1.42 (2H, m), 1.31 (2H, m), 1.29 (4H, m), 1.27 (6H, m), 1.25 (4H, m), 0.9 (3H, s).

¹³C NMR (125 MHz, CDCl₃) δ 173.1 (C═O), 121.9 (C), 110.1 (CH), 88.5 (CH), 83.0 (CH), 51.75 (OCH₃), 33.1 (CH₂), 32.5 (CH₂), 32.1 (CH₂), 31.1 (CH), 26.5 (2CH₃), 25.7 (CH₂), 23.7 (CH₂), 22.7 (2CH₂), 14.1 (CH₃).

Compound 7d

¹H NMR (500 MHz, CDCl₃) δ5.24 (1H, d, ³J=3.8), 4.2 (1H, dd, J=4.5, 3.8), 3.8 (1H, ddd, J=10.2, 7.9, 2.4), and 3.69 (3H, s, OCH₃), 2.66 (H, dd, J=16.9, 10.2), 2.32 (1H, dd, J=16.9, 4.5), 2.04 (1H, tt, J=10.2, 4.5), 1.42 (2H, m), 1.31 (2H, m), 1.29 (6H, m), 1.27 (6H, m), 1.25 (4H, m), 0.9 (3H, s).

¹³C NMR (125 MHz, CDCl₃) δ 173.1 (C═O), 121.9 (C), 110.1 (CH), 88.5 (CH), 83.0 (CH), 51.75 (OCH₃), 33.1 (CH₂), 32.5 (CH₂), 32.1 (CH₂), 31.1 (CH), 26.5 (2CH₃), 25.7 (CH₂), 23.7 (CH₂), 22.7 (2CH₂), 22.5 (CH₂), 14.1 (CH₃).

1.7 Compounds 8a, 8b, 8c, and 8d

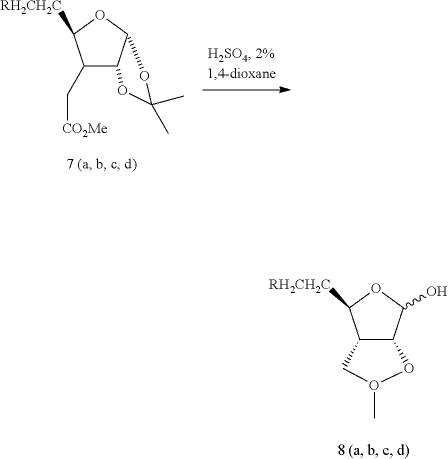

To a solution of compound 7 (a, b, c, d) (0.061 M) in p-dioxane (40 mL), 16 mL of aqueous sulfuric acid (2%) was added. The mixture was refluxed for 3 h. After cooling, 250 mL Et₂O were added, the organic phases were washed with water, saturated NaHCO₃ solution, dried over MgSO₄ and concentrated under reduced pressure. The crude products were purified by column chromatography on silica gel (Hexane:EtOAc 1:1) to yield compounds 8 as mixtures of epimers (5α:5β=1:2).

Compound 8a

¹H NMR (500 MHz, CDCl₃) δ6.07 (1H, d, J=3.8), 4.69 (1H, d, J=4.2), 3.73 (1H, d, J=6.0), and 3.65 (3H, s, OCH3), 2.4 (H, m), 2.38 (2H, m), 1.42 (2H, m), 1.31 (2H, m), 1.29 (4H, m), 0.88 (3H, s).

¹³C NMR (125 MHz, CDCl₃) δ 176.1 (C═O), 102.8 (CH), 93.2 (CH), 88.9 (CH), 32.8 (CH₂), 32.1 (CH₂), 31.0 (CH₂), 25.7 (CH₂), 22.7 (CH₂), 22.6 (CH), 14.1 (CH₃).

Compound 8b

¹H NMR (500 MHz, CDCl₃) δ6.07 (1H, d, J=3.8), 4.69 (1H, d, J=4.2), 3.73 (1H, d, J=6.0), and 3.65 (3H, s, OCH3), 2.4 (H, m), 2.38 (2H, m), 1.42 (2H, m), 1.31 (2H, m), 1.29 (2H, m), 1.29 (4H, m), 0.88 (3H, s).

¹³C NMR (125 MHz, CDCl₃) δ 176.1 (C═O), 102.8 (CH), 93.2 (CH), 88.9 (CH), 32.8 (CH₂), 32.1 (CH₂), 31.8 (CH₂), 31.0 (CH2), 25.7 (CH₂), 22.7 (CH₂), 22.6 (CH), 14.1 (CH₃).

Compound 8c

¹H NMR (500 MHz, CDCl₃) δ6.07 (1H, d, J=3.8), 4.69 (1H, d, J=4.2), 3.73 (1H, d, J=6.0), and 3.65 (3H, s, OCH3), 2.4 (H, m), 2.38 (2H, m), 1.42 (2H, m), 1.31 (2H, m), 1.29 (4H, m), 1.29 (4H, m), 0.88 (3H, s).

¹³C NMR (125 MHz, CDCl₃) δ 176.1 (C═O), 102.8 (CH), 93.2 (CH), 88.9 (CH), 32.8 (CH₂), 32.1 (CH₂), 31.8 (CH₂), 31.0 (CH2), 29.9 (CH₂), 29.3 (CH₂), 26.7 (CH₂), 22.6 (CH), 14.1 (CH₃).

Compound 8d

¹H NMR (500 MHz, CDCl₃) δ6.07 (1H, d, J=3.8), 4.69 (1H, d, J=4.2), 3.73 (1H, d, J=6.0), and 3.65 (3H, s, OCH3), 2.4 (H, m), 2.38 (2H, m), 1.42 (2H, m), 1.31 (2H, m), 1.29 (6H, m), 1.29 (4H, m), 0.88 (3H, s).

¹³C NMR (125 MHz, CDCl₃) δ 176.1 (C═O), 102.8 (CH), 93.2 (CH), 88.9 (CH), 32.8 (CH₂), 32.1 (CH₂), 31.8 (CH₂), 31.0 (CH₂), 29.9 (CH₂), 29.6 (CH₂), 29.3 (CH₂), 26.7 (CH₂), 22.6 (CH), 14.1 (CH₃).

1.8 Compounds 9a, 9b, 9c, and 9d

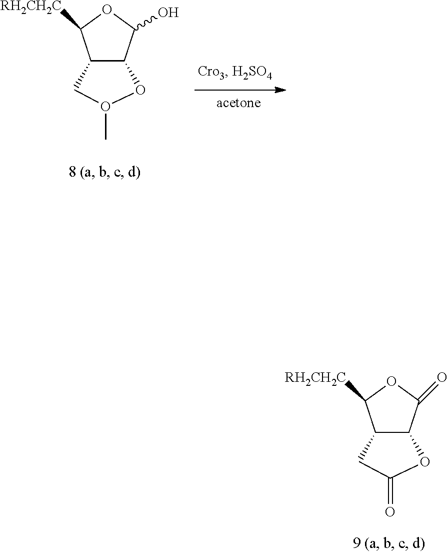

The Jones reagent (prepared from 26.7 g CrO₃ and 23.0 mL of conc. H₂SO₄ and water up to 100 mL) was added in drop wise manner to 0.054 mol L⁻¹ stirring solutions of compound 8 (a, b, c, d) in acetone (30 mL) till the mixtures acquired a permanent orange-brown color. Then, 50 mL of CH₂Cl₂ were added and stirring was continued for 10 min, when water (30 mL) was added. The organic phase was washed with saturated NaHCO₃ solution and with water, dried over MgSO₄ and concentrated under reduced pressure to yield compound 9 (a, b, c, d).

Compound 9a

¹H NMR (500 MHz, CDCl₃) δ4.76 (1H, d, J=3.8), 4.28 (1H, dd, J=10.8, 4.2), 3.31 (1H, m), 2.38 (2H, m), 1.53 (2H, m), 1.31 (2H, m), 1.25 (4H, m), 0.88 (3H, s).

¹³C NMR (125 MHz, CDCl₃) δ 176.1 (C═O), 173.1 (C═O), 86.5 (CH), 80.5 (CH), 35.0 (CH₂), 31.8 (CH₂), 31.4 (CH₂), 25.6 (CH₂), 22.7 (CH₂), 20.2 (CH) 14.1 (CH₃).

Compound 9b

¹H NMR (500 MHz, CDCl₃) δ4.76 (1H, d, J=3.8), 4.28 (1H, dd, J=10.8, 4.2), 3.31 (1H, m), 2.38 (2H, m), 1.53 (2H, m), 1.31 (2H, m), 1.29 (2H, m), 1.25 (4H, m), 0.88 (3H, s).

¹³C NMR (125 MHz, CDCl₃) δ 176.1 (C═O), 173.1 (C═O), 86.5 (CH), 80.5 (CH), 35.0 (CH₂), 31.8 (CH₂), 31.4 (CH₂), 29.3 (CH₂), 25.6 (CH₂), 22.7 (CH₂), 20.2 (CH) 14.1 (CH₃).

Compound 9c

¹H NMR (500 MHz, CDCl₃) δ4.76 (1H, d, J=3.8), 4.28 (1H, dd, J=10.8, 4.2), 3.31 (1H, m), 2.38 (2H, m), 1.53 (2H, m), 1.31 (2H, m), 1.29 (4H, m), 1.25 (4H, m), 0.88 (3H, s).

¹³C NMR (125 MHz, CDCl₃) δ 176.1 (C═O), 173.1 (C═O), 86.5 (CH), 80.5 (CH), 35.0 (CH₂), 31.8 (CH₂), 31.4 (CH₂), 29.6 (CH₂), 29.3 (CH₂), 25.6 (CH₂), 22.7 (CH₂), 20.2 (CH) 14.1 (CH₃).

Compound 9d

¹H NMR (500 MHz, CDCl₃) δ4.76 (1H, d, J=3.8), 4.28 (1H, dd, J=10.8, 4.2), 3.31 (1H, m), 2.38 (2H, m), 1.53 (2H, m), 1.31 (2H, m), 1.29 (6H, m), 1.25 (4H, m), 0.88 (3H, s).

¹³C NMR (125 MHz, CDCl₃) δ 176.1 (C═O), 173.1 (C═O), 86.5 (CH), 80.5 (CH), 35.0 (CH₂), 31.8 (CH₂), 31.4 (CH₂), 29.6 (2CH₂), 29.3 (CH₂), 25.6 (CH₂), 22.7 (CH₂), 20.2 (CH) 14.1 (CH₃).

1.9 Compounds 10a, 10b, 10c, and 10d

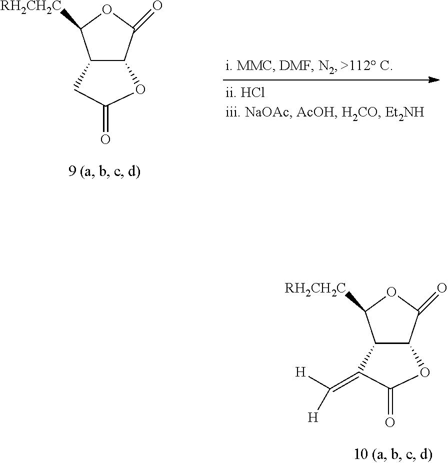

The bis-lactones 9 (a, b, c, d) (0.94 mmol) were dissolved in a 2.0 mol L⁻¹ solution of methyl methoxymagnesium carbonate in DMF (4.0 mL), under N₂ atmosphere. After 5 h under reflux (120° C.), the mixtures were poured into ice cold 6 molL⁻¹ HCl and Et₂O (1:5, 11 mL) and shaken until the precipitates were dissolved. The organic layers were then washed with water and dried over MgSO₄. The solvent was removed under reduced pressure affording the corresponding bis-γ-lactone carboxylic acids as colorless oils. Sodium acetate (0.2 g) was dissolved in acetic acid (8.0 mL) and mixed with a solution of formalin (6.0 mL) and diethylamine (2.0 mL). A portion of this solution (2.0 mL) was added to the bis-lactonic acids obtained and shaken vigorously until evolution of CO₂ ceased (2-3 min). The mixtures were heated on a steam bath for 5 min, cooled, and poured into water (20 mL) and ether (35 mL). The ether phases were washed with water and saturated NaHCO₃ solution and dried over MgSO₄. Evaporation of the ether afforded white solids, which were purified by column chromatography on silica gel with hexane/ethyl acetate 3:1, yielding compounds 10 (a, b, c, d).

Compound 10a

¹H NMR (500 MHz, CDCl₃) δ6.31 (1H, d, J=2.5), 5.79 (1H, d, J=2.2), 4.8 (1H, d, J=8.3), 4.32 (1H, m), 3.31 (1H, m), 1.53 (2H, m), 1.31 (2H, m), 1.25 (4H, m), 0.89 (3H, s).

¹³C NMR (125 MHz, CDCl₃) δ 173.7 (C═O), 170.6 (C═O), 136.1 (═C), 124.6 (═CH₂), 90.6 (CH), 88.3 (CH), 38.4 (CH), 31.9 (CH₂), 31.8 (2CH₂), 22.7 (CH₂), 14.1 (CH₃).

Compound 10b

¹H NMR (500 MHz, CDCl₃) δ6.31 (1H, d, J=2.5), 5.79 (1H, d, J=2.2), 4.8 (1H, d, J=8.3), 4.32 (1H, m), 3.31 (1H, m), 1.53 (2H, m), 1.31 (2H, m), 1.25 (4H, m), 1.29 (2H, m), 0.89 (3H, s).

¹³C NMR (125 MHz, CDCl₃) δ 173.7 (C═O), 170.6 (C═O), 136.1 (═C), 124.6 (═CH₂), 90.6 (CH), 88.3 (CH), 38.4 (CH), 31.9 (CH₂), 31.8 (2CH₂), 26.0 (CH₂), 22.7 (CH₂), 14.1 (CH₃).

Compound 10c

¹H NMR (500 MHz, CDCl₃) δ6.31 (1H, d, J=2.5), 5.79 (1H, d, J=2.2), 4.8 (1H, d, J=8.3), 4.32 (1H, m), 3.31 (1H, m), 1.53 (2H, m), 1.31 (2H, m), 1.25 (4H, m), 1.29 (4H, m), 0.89 (3H, s).

¹³C NMR (125 MHz, CDCl₃) δ 173.7 (C═O), 170.6 (C═O), 136.1 (═C), 124.6 (═CH₂), 90.6 (CH), 88.3 (CH), 38.4 (CH), 31.9 (CH₂), 31.8 (CH₂), 29.6 (2CH₂), 29.3 (CH₂), 26.0 (CH₂), 22.7 (CH₂), 14.1 (CH₃).

Compound 10d

¹H NMR (500 MHz, CDCl₃) δ6.31 (1H, d, J=2.5), 5.79 (1H, d, J=2.2), 4.8 (1H, d, J=8.3), 4.32 (1H, m), 3.31 (1H, m), 1.53 (2H, m), 1.31 (2H, m), 1.25 (4H, m), 1.29 (6H, m), 0.89 (3H, s).

¹³C NMR (125 MHz, CDCl₃) δ 173.7 (C═O), 170.6 (C═O), 136.1 (═C), 124.6 (═CH₂), 90.6 (CH), 88.3 (CH), 38.4 (CH), 31.9 (CH₂), 31.8 (CH₂), 29.6 (2CH₂), 29.3 (CH₂), 26.0 (CH₂), 22.7 (CH₂), 14.1 (CH₃).

1.10 Compounds 11a, 11 b, 11c, and 11d

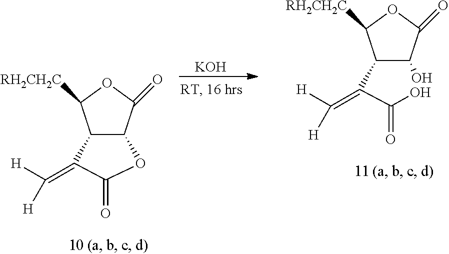

To a solution of compound 10 (a, b, c, d) (0.51 mmol) in 5 ml methanol was added potassium hydroxide (0.51 mmol). The reaction was heated under reflux for 4 h at 70° C., cooled to room temperature, acidified with 1 M potassium bisulphate and diluted with methanol. The resulting precipitate was filtered, washed with methanol and the filtrate reduced under vacuum. The crude product was flushed over silica to obtain 1 as a colorless solid (0.43 mmol, 85%).

Compound 11a

¹H NMR (500 MHz, CDCl₃) δ6.51 (1H, s), 5.79 (1H, s), 4.7 (1H, d, J=8.3), 4.56 (1H, m), 3.37 (1H, m), 1.69 (2H, m), 1.47 (2H, m), 1.26 (4H, m), 0.89 (3H, s).

¹³C NMR (125 MHz, CDCl₃) δ 176.9 (C═O), 170.4 (C═O), 135.1 (═C), 129.9 (═CH₂), 82.8 (CH), 68.9 (CH), 46.8 (CH), 34.9 (CH₂), 31.9 (CH₂), 31.8 (CH₂), 22.7 (CH₂), 14.1 (CH₃).

Compound 11 b

¹H NMR (500 MHz, CDCl₃) δ6.51 (1H, s), 5.79 (1H, s), 4.7 (1H, d, J=8.3), 4.56 (1H, m), 3.37 (1H, m), 1.69 (2H, m), 1.47 (2H, m), 1.26-1.29 (6H, m), 0.89 (3H, s).

¹³C NMR (125 MHz, CDCl₃) δ 176.9 (C═O), 170.4 (C═O), 135.1 (═C), 129.9 (═CH₂), 82.8 (CH), 68.9 (CH), 46.8 (CH), 34.9 (CH₂), 31.9 (CH₂), 29.3 (CH₂), 26.0 (CH₂), 22.7 (CH₂), 14.1 (CH₃).

Compound 11e

¹H NMR (500 MHz, CDCl₃) δ 6.51 (1H, s), 5.79 (1H, s), 4.7 (1H, d, J=8.3), 4.56 (1H, m), 3.37 (1H, m), 1.69 (2H, m), 1.47 (2H, m), 1.26-1.29 (8H, m), 0.89 (3H, s).

¹³C NMR (125 MHz, CDCl₃) δ 176.9 (C═O), 170.4 (C═O), 135.1 (═C), 129.9 (═CH₂), 82.8 (CH), 68.9 (CH), 46.8 (CH), 34.9 (CH₂), 31.9 (CH₂), 29.6 (CH₂), 29.3 (CH₂), 26.0 (CH₂), 22.7 (CH₂), 14.1 (CH₃).

Compound 11d

¹H NMR (500 MHz, CDCl₃) δ 6.51 (1H, s), 5.79 (1H, s), 4.7 (1H, d, J=8.3), 4.56 (1H, m), 3.37 (1H, m), 1.69 (2H, m), 1.47 (2H, m), 1.26-1.29 (10H, m), 0.89 (3H, s).

¹³C NMR (125 MHz, CDCl₃) δ 176.9 (C═O), 170.4 (C═O), 135.1 (═C), 129.9 (═CH₂), 82.8 (CH), 68.9 (CH), 46.8 (CH), 34.9 (CH₂), 31.9 (CH₂), 29.6 (2CH₂), 29.3 (CH₂), 26.0 (CH₂), 22.7 (CH₂), 14.1 (CH₃).

1.11 Compounds 12a, 12b, 12c, and 12d

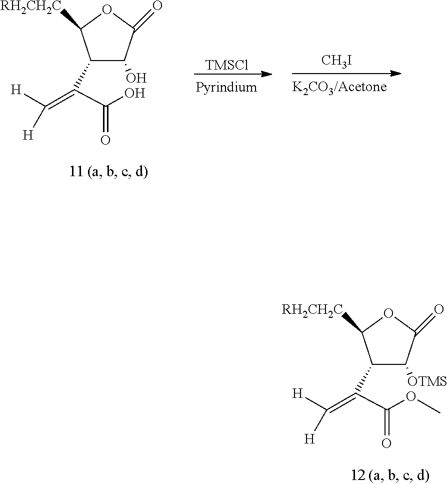

The compound 11 (a, b, c, d) was treated with tertbutyldimethylchlorosilane in pyridine, at room temperature, to provide the corresponding silylated products. Then the MeI (1.0 mL, 16.1 mmol) was added to a stirred mixture of the crude carboxylic acid (1.51 g) and K₂CO₃ (917 mg, 6.63 mmol) in acetone (15 mL). After 14 h, the resulting suspension was filtered through a Celite pad, and the filtrate was evaporated in vacuo. Purification of the residue (1.35 g) by column chromatography (silica gel 40 g, n-hexane/AcOEt 10:1→7:1) afforded 12 (935 mg, 79% for two steps).

Compound 12a

¹H NMR (500 MHz, CDCl₃) δ 6.51 (1H, s), 5.79 (1H, s), 4.7 (1H, d, J=8.3), 4.56 (1H, m), 3.79 (OCH₃, t), 3.37 (1H, m), 1.69 (2H, m), 1.47 (2H, m), 1.26 (4H, m), 0.98 (9H, s), 0.89 (3H, s), 0.21 (6H, s).

¹³C NMR (125 MHz, CDCl₃) δ 176.9 (C═O), 170.4 (C═O), 135.1 (═C), 129.9 (═CH₂), 82.8 (CH), 68.9 (CH), 52.5 (OCH₃), 46.8 (CH), 34.9 (CH₂), 31.9 (CH₂), 31.8 (CH₂), 25.9 (3CH₃), 22.7 (CH₂), 14.1 (CH₃), −2.3 (2CH₃).

Compound 12b

¹H NMR (500 MHz, CDCl₃) δ 6.51 (1H, s), 5.79 (1H, s), 4.7 (1H, d, J=8.3), 4.56 (1H, m), 3.79 (OCH₃, t), 3.37 (1H, m), 1.69 (2H, m), 1.47 (2H, m), 1.26-1.29 (6H, m), 0.98 (9H, s), 0.89 (3H, s), 0.21 (6H, s).

¹³C NMR (125 MHz, CDCl₃) δ 176.9 (C═O), 170.4 (C═O), 135.1 (═C), 129.9 (═CH₂), 82.8 (CH), 68.9 (CH), 52.5 (OCH₃), 46.8 (CH), 34.9 (CH₂), 31.9 (CH₂), 29.3 (CH₂), 25.9 (3CH₃), 26.0 (CH₂), 22.7 (CH₂), 14.1 (CH₃), −2.3 (2CH₃).

Compound 12c

¹H NMR (500 MHz, CDCl₃) δ 6.51 (1H, s), 5.79 (1H, s), 4.7 (1H, d, J=8.3), 4.56 (1H, m), 3.79 (OCH₃, t), 3.37 (1H, m), 1.69 (2H, m), 1.47 (2H, m), 1.26-1.29 (8H, m), 0.98 (9H, s), 0.89 (3H, s), 0.21 (6H, s).

¹³C NMR (125 MHz, CDCl₃) δ 176.9 (C═O), 170.4 (C═O), 135.1 (═C), 129.9 (═CH₂), 82.8 (CH), 68.9 (CH), 52.5 (OCH₃), 46.8 (CH), 34.9 (CH₂), 31.9 (CH₂), 29.6 (CH₂), 29.3 (CH₂), 26.0 (CH₂), 25.9 (3CH₃), 22.7 (CH₂), 14.1 (CH₃), −2.3 (2CH₃).

Compound 12d

¹H NMR (500 MHz, CDCl₃) δ 6.51 (1H, s), 5.79 (1H, s), 4.7 (1H, d, J=8.3), 4.56 (1H, m), 3.79 (OCH₃, t), 3.37 (1H, m), 1.69 (2H, m), 1.47 (2H, m), 1.26-1.29 (10H, m), 0.98 (9H, s), 0.89 (3H, s), 0.21 (6H, s).

¹³C NMR (125 MHz, CDCl₃) δ 176.9 (C═O), 170.4 (C═O), 135.1 (═C), 129.9 (═CH₂), 82.8 (CH), 68.9 (CH), 52.5 (0CH₃), 46.8 (CH), 34.9 (CH₂), 31.9 (CH₂), 29.6 (2CH₂), 29.3 (CH₂), 26.0 (CH₂), 25.9 (3CH₃), 22.7 (CH₂), 14.1 (CH₃), −2.3 (2CH₃).

1.12 Compounds 13a, 13b, 13c, and 13d

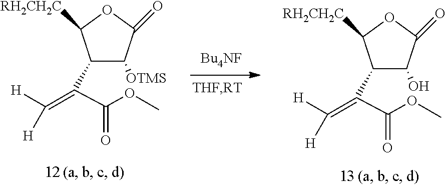

Bu₄NF in THF (1 M, 0.4 mL, 0.4 mmol) was added to a stirred mixture of TMS ether of compound 12 (a, b, d, d) (99 mg, 0.27 mmol) and AcOH (49 mg, 0.8 mmol) in THF (5.8 mL) at 0° C. After stirring at room temperature for 20 h, the reaction was quenched with saturated aqueous NaHCO₃ (10 mL), and the mixture was extracted with AcOEt (2×20 mL). The combined organic extracts were washed with brine (2×10 mL) and dried over anhydrous Na₂SO₄. Filtration and evaporation in vacuo furnished the crude product (100 mg), which was purified by column chromatography (silica gel 4.2 g, n-hexane/AcOEt 3:2) to 1 compound 13 (a, b, c, d).

Compound 13a

¹H NMR (500 MHz, CDCl₃) δ 6.42 (1H, s), 5.79 (1H, s), 4.7 (1H, d, J=8.3), 4.56 (1H, m), 3.79 (OCH₃, t), 3.37 (1H, m), 1.69 (2H, m), 1.47 (2H, m), 1.26 (4H, m), 0.89 (3H, s).

¹³C NMR (125 MHz, CDCl₃) δ 176.9 (C═O), 170.4 (C═O), 135.1 (═C), 129.9 (═CH₂), 82.8 (CH), 68.9 (CH), 52.5 (OCH₃), 46.8 (CH), 34.9 (CH₂), 31.9 (CH₂), 31.8 (CH₂), 22.7 (CH₂), 14.1 (CH₃).

Compound 13b

¹H NMR (500 MHz, CDCl₃) δ 6.42 (1H, s), 5.74 (1H, s), 4.7 (1H, d, J=8.3), 4.56 (1H, m), 3.79 (OCH₃, t), 3.37 (1H, m), 1.69 (2H, m), 1.47 (2H, m), 1.26-1.29 (6H, m), 0.89 (3H, s).

¹³C NMR (125 MHz, CDCl₃) δ 176.9 (C═O), 170.4 (C═O), 135.1 (═C), 129.9 (═CH₂), 82.8 (CH), 68.9 (CH), 52.5 (OCH₃), 46.8 (CH), 34.9 (CH₂), 31.9 (CH₂), 29.3 (CH₂), 26.0 (CH₂), 22.7 (CH₂), 14.1 (CH₃).

Compound 13c

¹H NMR (500 MHz, CDCl₃) δ 6.42 (1H, s), 5.74 (1H, s), 4.7 (1H, d, J=8.3), 4.56 (1H, m), 3.79 (OCH₃, t), 3.37 (1H, m), 1.69 (2H, m), 1.47 (2H, m), 1.26-1.29 (8H, m), 0.89 (3H, s). ¹³C NMR (125 MHz, CDCl₃) δ 176.9 (C═O), 170.4 (C═O), 135.1 (═C), 129.9 (═CH₂), 82.8 (CH), 68.9 (CH), 52.5 (0CH₃), 46.8 (CH), 34.9 (CH₂), 31.9 (CH₂), 29.6 (CH₂), 29.3 (CH₂), 26.0 (CH₂), 22.7 (CH₂), 14.1 (CH₃).

Compound 13d

¹H NMR (500 MHz, CDCl₃) δ 6.42 (1H, s), 5.74 (1H, s), 4.7 (1H, d, J=8.3), 4.56 (1H, m), 3.79 (OCH₃, t), 3.37 (1H, m), 1.69 (2H, m), 1.47 (2H, m), 1.26-1.29 (10H, m), 0.89 (3H, s).

¹³C NMR (125 MHz, CDCl₃) δ 176.9 (C═O), 170.4 (C═O), 135.1 (═C), 129.9 (═CH₂), 82.8 (CH), 68.9 (CH), 52.5 (OCH₃), 46.8 (CH), 34.9 (CH₂), 31.9 (CH₂), 29.6 (2CH₂), 29.3 (CH₂), 26.0 (CH₂), 22.7 (CH₂), 14.1 (CH₃).

### Example 2

### The Isolation of Avenaciolide Derivatives from Neosartoya fischeri

Avenaciolide derivatives may also be isolated from Neosartoya fischeri according to steps described by Yang et al (Planta Med (2010) 76, 1701-1705).

Briefly, the mycelium of Neosartoya fischeri was inoculated into a mixture of 10 g Bacto™ malt extract (Becton Dickinson) and 500 mL distilled water, and then let fermenting at 25-30° C. for about 30 days. The fermented broth (about 108 L) was then filtered and partitioned 3 times with 50 L ethyl acetate, then concentrated under vacuum to dryness (0.6 g). Subsequently, the residue was redissolved in 25 mL methanol, and loaded onto a Sephadex LH-20 column (3 cm i.d.×65 cm) eluted with MeOH in a flow rate of 2.5 mL/min. Each fraction (25 mL) collected was checked by TLC using EtOAc/acetic acid/water (85:10:10, v/v/v) for development, and observed under UV 254 nm. Subfractions were combined and subsequently purified by HPLC, which gave rise to 4 Avenaciolide derivatives (i.e., compound 10d, 13d, 14 and 15) respectively having the following structures,

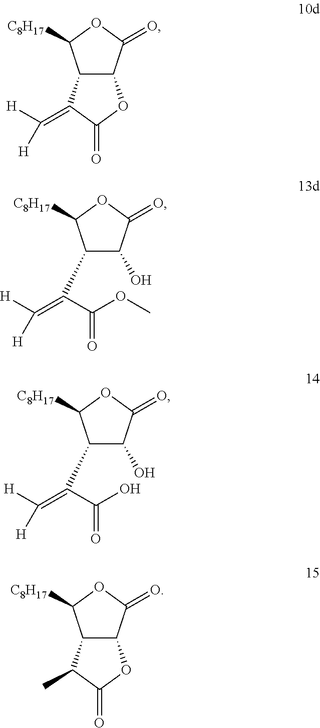

### Example 3

### Avenaciolide Derivatives Suppress the Growth of Gram-Positive Bacteria

In this example, the inhibitory effect of the isolated avenaciolide derivatives of example 2 (i.e., compounds 10d, 13d, 14, and 15) on the growth of gram-positive bacteria, including MRSA, were tested; and the results are summarized in Table 1.

TABLE 1
Anti-bacteria Activity of The Compounds of Example 2

| | MIC (μg mL⁻¹) | | | |
|---|---|---|---|---|
| | Com- | Com- | Com- | |
| | pound 10d | pound 13d | pound 14 | fosfomycin^{[a]} |
| S. aureus | 16 | 32 | 256 | 4 |
| ATCC 29213 | | | | |
| S. aureus | 32 | 16 | 256 | 64 |
| ATCC 33592^{[b]} | | | | |
| B. subtilis | 64 | 32 | 256 | 128 |
| ATCC 23857 | | | | |
| E. coli | 128 | 128 | no | 64 |
| ATCC 25922 | | | activity | |
| A. baumannii | 256 | 256 | no | 256 |
| 17978 | | | activity | |

^{[a]}fosfomycin was used as a reference drug/
^{[b]}This is a MRSA strain.

As is evident from Table 1, both compounds 10d and 13d were more effective in suppressing the growth of gram-positive bacteria, including Staphylococcus aureus, methicillin-resistant Staphylococcus aureus (MRSA), Bacillus subtilis, and E. Coli, than that of a gram-negative bacteria, such as Acinetobacter baumannii, with the MIC for gram-positive bacteria being about 16-32 μg/mL, and 256 μg/mL for gram-negative bacteria. Further, neither compound 14, nor compound 15 possessed any anti-bacteria activity. This observation was further confirmed by TEM photographs.

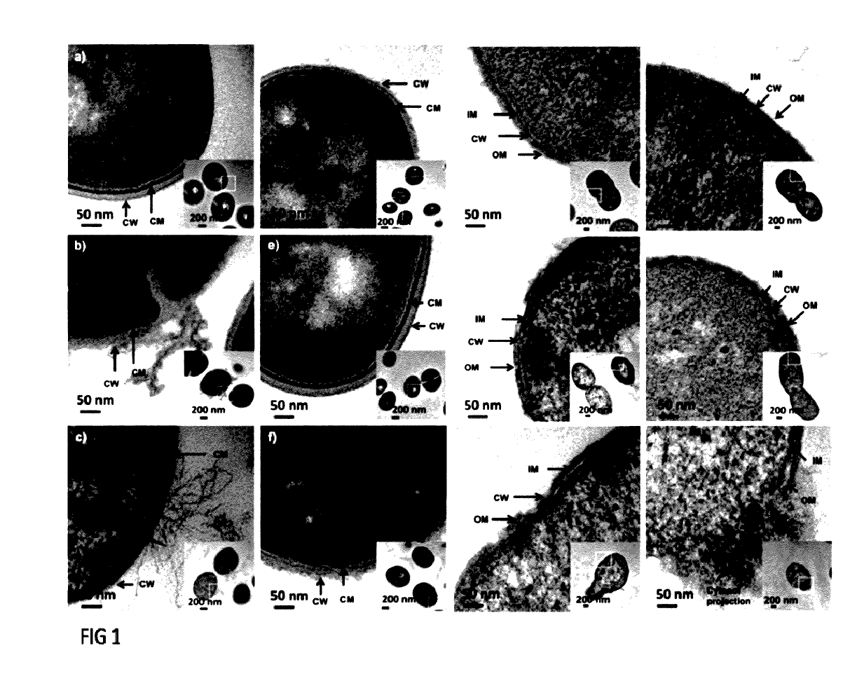

Referring to photographs in FIG. 1, compared to the normal healthy cells, in which the cell walls appeared to be round and smooth (FIG. 1 (a)), the cell walls were ruptured if treated with the compound 10d or 13d (128 μg/mL) (FIGS. 1(b) and 1(c)). However, the cell walls for gram-negative bacteria were not affected by such treatment (see FIGS. 1(g), 1(h) and 1(i)), in which FIG. 1(g) is the photograph of un-treated Acinetobacter baumannii, and FIGS. 1(h) and 1(i) are photographs of A. baumannii respectively taken after being treated with the compound 10d and 13d for an hr.

FIGS. 1(d) and 1(j) are photographs of MRSA and A. baumannii respectively taken after being treated with the compound 14 for an hr, in which the cell wall structure of MRSA was slightly affected by the treatment, whereas baumannii was completely unaffected.

As to the effect of compound 15, neither MRSA nor A. baumannii was affected by the treatment of compound 15 (see FIGS. 1(e) and 1(k)). Fosfomycin was also used as a positive control, in which both MRSA and A. baumannii became ruptured after being treated with fosfomycin (64 μg/mL).

Comparing the structure differences between the compounds that underwent growth inhibition test, it seems that α,β-unsaturated carbonyl is a necessary moiety for an avenaciolide derivative to possess any anti-bacteria activity. Take the compound 15 as an example, it lacks α,β-unsaturated carbonyl in its structure, and as expected, no antibacterial activity either. Accordingly, it is reasonable to hypothesize that both compounds 10d and 13d inhibit bacterial growth by suppressing the activity of the enzyme MurA (UDP-NAG-enolpyruvyl transferas) that catalyzes the cell wall peptidoglycan synthesis, which is critical for cell survival. Accordingly, the compounds of example 2 were subject to further enzymatic test to see if any of them was capable of suppressing the activity of MurA enzyme.

Various types of MuraA enzymes, including those from E. Coli, wild type MRSA^{WT}, and MRSA^{C119D} mutant strain, were treated with any of the compounds 10d, 13d, or 14, as well as fosfomycin, in which MRSA^{WT} represents wild type MurA, and MRSA^{C119D} mutant strain represents MRSA strain in which the 119^{th} amino acid residue, cysteine (C), is replaced by aspartic acid (D). Results are summarized in Table 2.

TABLE 2
Inhibitory Effect of The Compounds
of Example 2 on MurA Activity

| | IC₅₀ (μM) | | | |
|---|---|---|---|---|
| | Com- | Com- | Com- | |
| | pound 10d | pound 13d | pound 14 | fosfomycin^{[a]} |
| E coli MurA | 0.9 ± | 2.8 ± | 10.8 ± | 0.4 ± |
| | 1.11^{[b]} | 1.22^{[b]} | 1.13^{[b]} | 1.16^{[b]} |
| MRSA MurA^{WT} | 8.3 ± | 6.7 ± | 71 ± | 1.6 ± |
| | 1.22^{[b]} | 1.17^{[b]} | 1.17^{[b]} | 1.12^{[b]} |
| MRSA MurA^{C119D} | 21.5 ± | 7.9 ± | No | No |
| | 1.33^{[b]} | 1.24^{[b]} | activity | activity |

^{[a]}fosfomycin was used as a reference drug.
^{[b]}The standard deviation of the IC₅₀ value was calculated based on 5 replicates.

It is well known that MurA from the MRSA^{C119D} mutant strain is resistant to fosfomycin, accordingly, as expected in data summarized in Table 2, fosfomycin failed to suppress MurA of MRSA^{C119D} mutant strain; conversely, the compounds 10d, 13d, and 14 all exhibited inhibitory effect toward MurA of E. Coli, with IC₅₀ values between 0.9-10.8 μM. Similarly, the compounds 10d and 13d were also capable of inhibiting MurA activities of wild type MRSA and MRSA^{C119D} mutant strain, with IC₅₀ values between 0.9-21.5 μM. As to compound 14, it could slightly inhibit MurA of MRSA (IC₅₀ was about 71 μM), but was in-effective toward that of MRSA^{C119D} mutant strain.

### Example 4

### Avenaciolide Derivatives Suppress the Growth of Macrophages

In this example, the effect of the compounds of example 2 on the growth of mammalian cells were tested.

In general, mice macrophages were treated with various concentrations of the compounds of example 2 for 24 hrs, the viability of cells were then analyzed by fluorescence analysis in accordance with procedures described in “Materials and Methods” section.

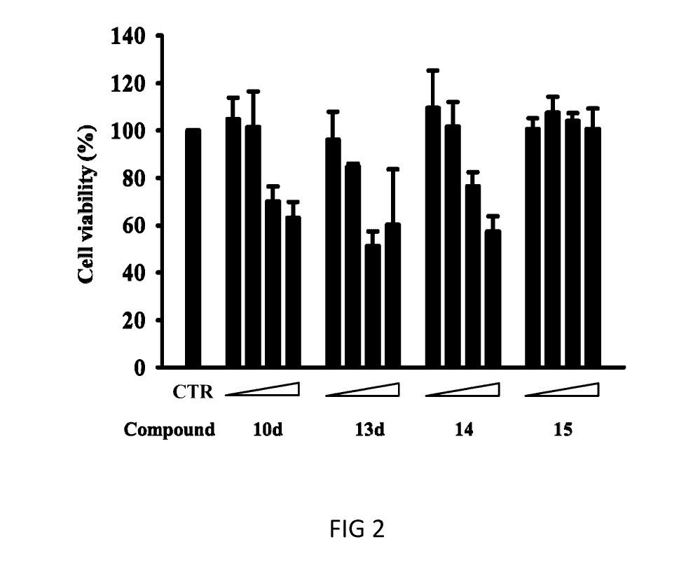

As depicted in FIG. 2, the growth of macrophages was completely un-affected by the treatment of compound 15; and was slightly affected by low concentrations of compounds 10d, 13d, and 14 (12.5 or 25 μM). Only when the concentration of the compounds 10d, 13d, and 14 reached 50 or 100 μM, were there significant growth inhibition on the macrophages.

Taken together, the compound of the present disclosure (e.g., the compound 10d or 13d) may suppress the growth of gram-positive bacteria, including Staphylococcus aureus, methicillin-resistant Staphylococcus aureus (MRSA), and Bacillus subtilis by suppressing the activity of MurA responsible for peptidoglycan synthesis on the cell wall, thus the compound of the present disclosure possesses antibacterial activity by disrupting the integrity of the cell wall structure. Further, the capability of the compounds of the present disclosure suppress the growth of gram-positive bacteria at low concentrations without affecting the activity of immune cells (e.g., macrophages) rendering the compounds of the present disclosure potential lead compounds for the development of next generation antibiotic for treating diseases associated with infections caused by gram-positive bacteria, which include but are not limited to, Staphylococcus aureus, methicillin-resistant Staphylococcus aureus (MRSA), and Bacillus subtilis. Most importantly, the present disclosure offer solutions to current clinical issue of lacking effective medicament for combating infections caused by drug-resistant bacteria.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

What is claimed is:

1. A method of treating a disease associated with a gram-positive bacterial infection in a subject, comprising administering to the subject, an effective amount of a derivative of avenaciolide having the structure of formula (I) or (II), so as to alleviate or ameliorate the symptoms of the disease, 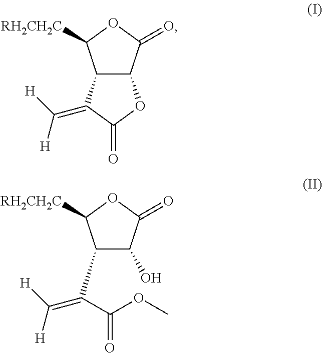
wherein, R is C₂₋₁₀ alkyl or C₂₋₁₀ alkenyl.

2. The method of claim 1, wherein R is n-hexyl.

3. The method of claim 1, wherein the gram-positive bacteria is any of Bacillus anthracis, Bacillus subtilis, Bacillus cereus, Corynebacterium diptheriae, Clostridium tetani, Clostridium botulinum, Clostridium perfringes, Clostridium difficile, Clostridium scindens, Enterococcim Streptococcus viridians, Enterococcus faecalis, Erysipelothrix rhusiopathiae, Escherichia Coli, Listeria monocytogens, Propionbacterium acnes, Rhodococcus equi, Staphylococcus agalactiae, Staphylococcus aureus, Staphylococcus epidermidis, Staphylococcus pneumonia, Staphylococcus pyrogens, or Staphylococcus saprophyticus.

4. The method of claim 3, wherein the Staphylococcus aureus is a methicillin-resistant Staphylococcus aureus (MRSA).

5. The method of claim 3, wherein the Enterococcus faecalis is a vancomycin resistant Enterococcus faecalis.

6. The method of claim 1, wherein the disease is pneumonia, sepsis, cornea infection, skin infection, an infection in the central neuron system, or a toxic shock syndrome.

7. The method of claim 1, wherein the subject has skin abscess, furuncle or skin boil.

8. The method of claim 1, further comprising administering to the subject an antibiotic before, concurrently with, or after the administration of the derivative of avenaciolide having the structure of formula (I) or (II).

9. The method of claim 8, wherein the antibiotic is selected from the group consisting of, acumycin, ampicillin, amoxycillin, amphotericins, antimycins, anglomycin, avermectins, azithromycin, boromycin, carbomycins, carbapenem, ceftazidime, cethromycin, chloramphenicol, chalcomycin, ciprofloxacin, concanamycins, cirramycin, clarithromycin, colistin, cycloxacillin, daptomycin, desmethyl azithromycin, desertomycins, dihydropikromycin, dirithromycin, doxycycline, enramycin, erythromycin, flurithromycin, flumequin gentamycin, juvenimicins, kujimycins, lankamycins, lincomycin, litorin, leucomycins, megalomicins, meropenem, methymycin, midecamycins, mycinamicin I, mycinamicin II, mycinamicin III, mycinamicin IV, mycinamicin V, mycinamicin VI, mycinamicin VII, mycinamicin VIII, narbomycin, neoantimycin, neomethymycin, netilmicin, neutromycin, niddamycins, norfioxacin, oleandomycins, oligomycins, ossamycin, oxacillin, oxolinic acid, penicillin, pikromycin, piperacillin, platenomycins, rapamycins, relomycin, rifamycins, rosaramicin, roxithromycin, virginiamycin, spiramycin, sporeamycin, staphococcomycin, streptomycin, sulfamethoxazole, swalpamycin, telithromycin, teicoplanin, timentin, tobramycin, ticarcillin, trimethoprim, tetracyclin, zlocillin, and/or a combination thereof.

10. A method for suppressing the growth of a gram-positive bacteria comprising contacting the gram-positive bacteria with a compound of formula (I) or (II) for a sufficient period of time, 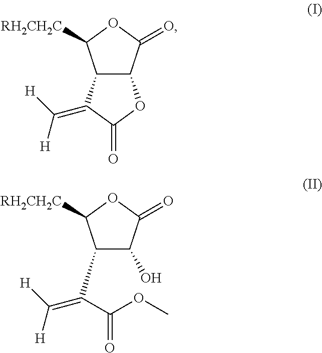
wherein, R is a C₂₋₁₀ alkyl or C₂₋₁₀ alkenyl.

11. The method of claim 10, wherein R is n-hexyl.

12. The method of claim 10, wherein the gram-positive bacteria is any of Bacillus anthracis, Bacillus subtilis, Bacillus cereus, Corynebacterium diptheriae, Clostridium tetani, Clostridium botulinum, Clostridium perfringes, Clostridium difficile, Clostridium scindens, Enterococcim Streptococcus viridians, Enterococcus faecalis, Erysipelothrix rhusiopathiae, Escherichia Coli, Listeria monocytogens, Propionbacterium acnes, Rhodococcus equi, Staphylococcus agalactiae, Staphylococcus aureus, Staphylococcus epidermidis, Staphylococcus pneumonia, Staphylococcus pyrogens, or Staphylococcus saprophyticus.

13. The method of claim 12, wherein the Staphylococcus aureus is a methicillin-resistant Staphylococcus aureus (MRSA).

14. The method of claim 12, wherein the Enterococcus faecalis is a vancomycin resistant Enterococcus faecalis.

15. A method of producing a compound of formula (I) or (II), 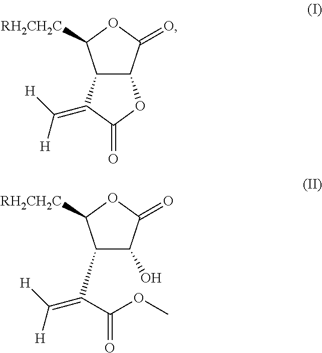
wherein R is a C₂₋₁₀ alkyl or C₂₋₁₀ alkenyl, and the method comprises:
(1) using diacetone D-glucose as a starting material to produce compound 5;
(2) allowing the compound 5 to react with a Wittig reagent to produce compound 6;
(3) reducing the compound 6 to give compound 7;
(4) allowing the compound 7 to react with 1,4-dioxane to produce compound 8 in an acidic condition;
(5) oxidizing the compound 8 to give compound 9;
(6) allowing the compound 9 to react with ethylene diamine to produce the compound of formula (I);
(7) allowing the compound of formula (I) to undergo a ring-opening reaction in an alkaline condition so as to produce compound 11;
(8) allowing the compound 11 to react with trimethylchlorosilane and methanol in the presence of a tertially amine so as to produce compound 12;
(9) hydrolyzing the compound 12 in the presence of a quaternary ammonium compound to produce the compound of formula (II);
wherein the compounds 5, 6, 7, 8, 9, 10, 11, and 12 respectively have the following structures,

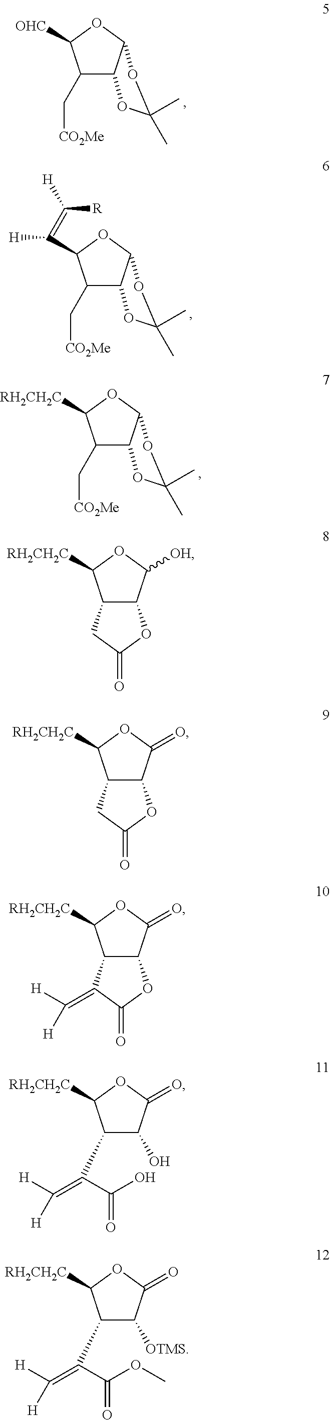

16. The method of claim 15, wherein the step (1) comprises,
(a) let diacetone D-glucose react with pyridiunm dichromate to produce compound 2;
(b) let the compound 2 react with tri-ethyl phosphonoacetate to produce compound 3;
(c) allowing the compound 3 to undergo a ring-opening reaction to generate compound 4; and
(d) let the compound 4 react with an oxidizing agent and subsequently with a reducing agent to produce the compound 5;
wherein, the compounds 2, 3, and 4 respectively have the following structures,

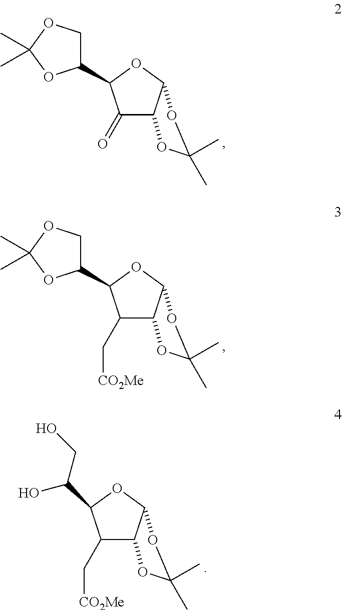

17. The method of claim 15, wherein the Wittig reagent in the step (2) is C₄₋₁₂ alkyltriphenylphosphonium bromide or C₄₋₁₂ alkenylltriphenylphosphonium bromide.

18. The method of claim 17, wherein the Wittig reagent is hexyltriphenylphosphonium bromide.

19. The method of claim 15, wherein the step (6) comprises,
(e) let the compound 9 react with methyl methoxymagnesium carbonate in an inert environment; and
(f) allowing the product of the step (e) to react with an acid and subsequently with diethyl amine to produce the compound of formula (I).

20. The method of claim 15, wherein the step (7) is performed in the presence of potassium hydroxide or sodium hydroxide.

21. The method of claim 15, wherein the quaternary ammonium compound in the step (9) is tetrabutylammonium fluoride, tetrabutylammonium chloride, tetrabutylammonium bromide, tetrabutylammonium iodide, tetrabutylammonium perchlorate, tetrabutylammonium hexafluorophosphate, or tetrabutylammonium acetate.
